(19) United States
(12) Patent Application Publication
(10) Pub. No.: US 2009/0018161 A1
(43) Pub. Date: Jan. 15, 2009

(54) METHOD FOR PROMOTING GASTROINTESTINAL FUNCTION AND METHOD FOR SCREENING FOR PROMOTER OF GASTROINTESTINAL FUNCTION

(75) Inventors: Tatsuro Tanaka, Kawasaki-shi (JP); Hisayuki Uneyama, Kawasaki-shi (JP); Shinichi Fujita, Kawasaki-shi (JP); Saori Ogawa, Kawasaki-shi (JP); Yusuke Amino, Kawasaki-shi (JP); Seiji Shiraishi, Kawasaki-shi (JP); Koichi Fujita, Kawasaki-shi (JP); Tatsuhiro Yamada, Kawasaki-shi (JP); Takashi Yamamoto, Kawasaki-shi (JP); Takeshi Aihara, Kawasaki-shi (JP); Manabu Suzuki, Kawasaki-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: AJINOMOTO CO. INC., Tokyo (JP)

(21) Appl. No.: 12/114,125

(22) Filed: May 2, 2008

Related U.S. Application Data
(63) Continuation-in-part of application No. PCT/JP2006/322411, filed on Nov. 2, 2006.
(60) Provisional application No. 60/738,561, filed on Nov. 22, 2005.

(30) Foreign Application Priority Data
Nov. 4, 2005 (JP) 2005-320827

(51) Int. Cl. A61K 31/47; A61K 31/16; A61K 31/164; A61K 31/343; A61K 31/4439; G01N 33/53; A61P 1/00; C12Q 1/02; A61K 31/221; A61K 31/357; A61K 31/44

(52) U.S. Cl. 514/311; 514/608; 514/622; 514/469; 514/466; 514/357; 514/534; 514/340; 435/29; 435/7.2

(57) ABSTRACT

The present invention provides a more effective gastrointestinal function promoter. The present invention also provides a method for the prophylaxis or improvement of functional gastrointestinal disorders, a method for appetite regulation and the like. The present invention also provides a method for screening for a substance capable of promoting a gastrointestinal function. The present invention provides a gastrointestinal function promoter containing a T1R agonist as an active ingredient and a method for the prophylaxis or improvement of functional gastrointestinal disorders, a method for appetite regulation and the like by administering a T1R agonist. The present invention also provides a method of screening for a substance capable of promoting a gastrointestinal function which method uses a cell expressing T1R receptor.

### CROSS REFERENCES TO RELATED APPLICATIONS

The present application is a continuation-in-part of International Patent Application No. PCT/JP2006/322411, filed on Nov. 2, 2006, and claims priority to Japanese Patent Application No. 2005-320827, filed on Nov. 4, 2005, and U.S. Provisional Application No. 60/738,561, filed on Nov. 22, 2005, all of which are incorporated herein by reference in their entireties.

### BACKGROUND OF THE INVENTION

1. Field of the Invention

The present invention relates to gastrointestinal function promoters, such as agents and methods for the prophylaxis or improvement of functional gastrointestinal disorders. More particularly, the present invention relates to agents and methods for the prophylaxis or improvement of functional gastrointestinal disorders (FGIDs), particularly, upper gastrointestinal dysfunctions such as functional dyspepsia (FD) (e.g., abdominal pain, heavy stomach, heartburn and the like), gastroesophageal reflux disease (GERD), gastroparesis, post-operative ileus, constipation, and the like. Moreover, the present invention relates to appetite regulators and methods for regulating the appetite. The present invention further relates to methods for screening for a substance capable of promoting gastrointestinal function.

2. Discussion of the Background

Even with the advancement in endoscopic diagnosis, there are many cases where a complaint of the upper gastrointestinal symptoms such as upper abdominal pain, discomfort, postprandial heavy stomach, nausea, vomiting and the like cannot be fully explained. Such a condition where a complaint of gastrointestinal symptom is reported but no organic disease is found by a general checkup including endoscopic examination, and no finding to elucidate the symptom is available is referred to as an FD (functional dyspepsia: non-ulcer dyspepsia (NUD): upper abdominal dyspepsia).

According to The American Gastroenterological Association, FD is defined to be a pathology where organic diseases such as peptic ulcer and cancer symptoms are not observed, but upper abdominal dyspepsia continues for 4 weeks or longer, such as feeling of abdominal distention, nausea.vomiting, upper abdominal pain, anorexia, abnormal bowel movement and the like, based on the retention of contents in the stomach. On the other hand, in Japan, such a case has been determined to be “upper abdomen gastrointestinal complaint associated with chronic gastritis” irrespective of organic findings, and, in clinical situations, diagnosed conventionally as “gastritis” or “chronic gastritis”. Currently, the subtype of FD includes an ulcer type, a gastrointestinal dysmotility type and non-specific type, which include conventional gastroatonia, nervous dyspepsia and gastrointestinal neurosis.

Even in cases where an organic disease (reflux esophagitis, peptic ulcer, acute gastritis, gastrointestinal cancer, pancreas.biliary disease, etc.) is clearly observed, abdominal pain, abdominal discomfort, postprandial heavy stomach, nausea.vomiting and the like are also found. Accordingly, there is an urgent need for the improvement of such discomfortable feeling to ensure a better quality of life (“QOL”) of patients. When FD is joined with lower abdomen dyspepsia such as defecation difficulty, unrelieved feeling after defecation, abdominal pain, feeling of abdominal distention and the like due to constipation, about 30% to 50% of the total population of Japan is assumed to have experienced some dyspepsia, of which one-third is said to have actually visited medical institutions. It is considered that the onset of abdominal dyspepsia is influenced by sex, aging, stress, and being overweight due to a Western-style diet, and abdominal dyspepsia is also a representative disease of the modern society, along with the lifestyle-related diseases. Being such a serious medical problem, the etiology of the dyspepsia is only a suggested relationship with various diseases (chronic gastritis, diabetes, overweight, constipation, etc.), where its developmental mechanism is not more than a mere suggestion of low gastrointestinal motility function. Taken together with the fact that the gastrointestinal motility function is degraded only in 30% of the total actual FD patients, it is clear that the developmental mechanism of FD has not been elucidated completely.

In addition, many of the patients suffering from progressive brain degenarative diseases such as Parkinson's disease, Huntington chorea, olivopontocerebellar atrophy and the like, cerebral apoplexy and the like concurrently develop gastrointestinal motility dysfunction. Thus, it is considered that the QOL needs to be improved by enhancing the gastrointestinal motility function. These patients are considered to include many who cannot report dyspepsia by themselves because of language disorder, consciousness disorder, and the like. Therefore, care for removing sensory abnormality such as dyspepsia and the like, which is performed simultaneously with care of organic dysfunction, should lead to the improvement of the QOL in a real sense.

For the treatment of FD, motility improvers such as 5-HT 4 receptor agonists and the like, and the like have been used. For example, cisapride and metoclopramide promote gastrointestinal motility, and have been used for the treatment of symptoms such as chronic gastritis, feeling of abdominal distention, reflux esophagitis, abdominal dyspepsia and pseudoileus, and the like. However, it has been clarified that metoclopramide shows side effects including extrapyramidal symptoms associated with the action on dopamine D2 receptor in the central nervous system, and cisapride not only shows Parkinson symptoms but also side effects on the circulatory system, such as QT prolongation and the like. While mosapride and the like are used, the effect is sometimes insufficient. Additionally, side effects such as feeling of abdominal distention, stomach pain and the like appear. For the treatment of gastroesophageal reflux disease (GERD) type, H2 antagonists and proton pump inhibitors are used. However, for long-term administration, a regular checkup is necessary since its safety is not certain. It is therefore difficult to seek, while ensuring sufficient safety, a treatment effect from these existing pharmaceutical agents.

As appetite regulators, fenfluramine and phentermine, which act on the central nerve as a point of action, sibutramine, mazindol and the like are known. However, dry mouth, constipation, sweating, palpitation, and the like may be seen as side effects, and an appetite regulator with a fewer side effects has been desired. Recently, it is considered that gastrointestinal motility plays a critical role in the rate of consumption of foods, digestion, and absorption of nutrients (Obes. Surg. 2007, 17(10): 1399-1407), and it is reported that regulation of gastric emptying is one of the targets of regulating the appetite (J. Clin. Endocrinol. Metab. 2006, 91(9): 3296-3302).

In the meantime, it has been clarified in recent years that the signal transduction of taste is performed via various receptors. As the taste receptors in mammals, two families of G protein binding type receptors called T1R and T2R have been found (see, WO2003/001876, WO2005/015158, WO2005/041684, WO2006/084184, WO2006/084186, WO2006/084246). They are specifically expressed in the taste cells in the tongue of humans and rodents, and are involved in the reception of sweet, umami, and bitter from five basic tastes. T1R is a receptor that recognizes sweet and umami, and T2R forms a family relating to bitter taste reception. As to T1R, T1R1, T1R2 and T1R3 are known subunits. When T1R2 and T1R3 form a hetero dimer, it responds to natural and artificial sweeteners and functions as a sweetness receptor. When T1R1 and T1R3 are bonded, they respond to umami substances such as amino acid and the like. By activation of these taste receptors, an unknown transmitter is released from a taste cell, which stimulates the taste nerve, and the taste signal is transmitted to the brain. However, the presence and function of them in the digestive system are not known. Furthermore, method of regulating an appetite which comprises administering an effective amount of a T1R agonist is not known.

Thus, there remains a need for agents and methods for the prophylaxis or improvement of functional gastrointestinal disorders. More particularly, there also remains a need for agents and methods for the prophylaxis or improvement of functional gastrointestinal disorders (FGIDs), particularly, upper gastrointestinal dysfunctions such as functional dyspepsia (FD) (e.g., abdominal pain, heavy stomach, heartburn and the like), gastroesophageal reflux disease (GERD), gastroparesis, post-operative ileus, constipation, and the like. There also remains a need for appetite regulators and methods for regulating the appetite. There also remains a need for methods for screening for a substance capable of promoting gastrointestinal function.

### SUMMARY OF THE INVENTION

Accordingly, it is one object of the present invention to provide novel gastrointestinal function promoters.

It is another object of the present invention to provide novel agents for the prophylaxis or improvement of functional gastrointestinal disorders.

It is another object of the present invention to provide novel appetite regulators.

It is another object of the present invention to provide novel compositions for the prophylaxis or improvement of functional gastrointestinal disorders.

It is another object of the present invention to provide novel compositions for appetite regulation.

It is another object of the present invention to provide novel methods of promoting a gastrointestinal function.

It is another object of the present invention to provide novel methods for the prophylaxis or improvement of a functional gastrointestinal disorder such as an upper gastrointestinal dysfunction, functional dyspepsia, a gastroesophageal reflux disease and the like.

It is another object of the present invention to provide novel methods of regulating the appetite.

It is another object of the present invention to provide novel methods of screening for a substance capable of promoting the gastrointestinal function.

These and other objects, which will become apparent during the following detailed description, have been achieved by the inventors' study of the T1R receptor. As a result, they have found that the T1R receptor is present in the intragastric mucous membrane layer and the small intestine mucous membrane layer, and that the T1R receptor is expressed in the gastrointestinal hormone-producing cells, particularly gastrin-producing cells.

To be specific, in the stomach and small intestine, the endocrine cells of gastrointestinal hormone are positive. Particularly, gastrin-producing cell, which is a gastrointestinal hormone, is positive in the pyloric vestibular part of the stomach. In a stomach and small intestine mucous membrane sample, the expression of not only T1R1 mRNA but also T1R3 mRNA necessary for functional expression as taste receptor T1R1+T1R3 was observed. Based on such findings, the present inventors have found that they can provide a method of screening for an agonist and the like of a T1R receptor, and further a method of screening for a substance capable of promoting the gastrointestinal function, and the like, by utilizing gastrointestinal hormone-producing cells derived from human or animal, which express the T1R receptor.

Moreover, as a result of the study using an agonist to T1R receptor (hereinafter sometimes to be also simply referred to as “T1R agonist”), it has been found that the T1R receptor agonist promotes clearance of the contents of the stomach (hereinafter sometimes to be also simply referred to as “gastric emptying”). Gastric emptying is not exclusively controlled by stomach motility but also reflects the easiness of digestion in and after duodenum, toward which the contents are delivered. This is clear from the occurrence of delayed gastric emptying in the case of food containing much fat that resists digestion and absorption.

Accordingly, the present inventors have found that a digestion.absorption-promoting effect, i.e., gastrointestinal function-promoting action, can be obtained by a T1R agonist having a gastric emptying-promoting action. Promotion of gastric emptying reduces the feeling of abnormal abdominal distention in the early postprandial period and improves anorexia. Moreover, promotion of gastric emptying facilitates digestion and absorption, which in turn rapidly increases the blood nutrient concentration. Thus, an effect of increasing the feeling of satisfaction in the early postprandial period can also be expected. Based on the above-mentioned findings, the present inventors have found that T1R agonists are effective for promotion of gastrointestinal function and appetite regulation, and completed the present invention. That is, the present invention relates to a gastrointestinal function promoter and appetite regulator comprising a T1R agonist as an active ingredient, a method of screening for a substance capable of promoting the gastrointestinal function and the like.

Accordingly, the present invention includes at least the following.

(1) A gastrointestinal function promoting agent, comprising a T1R agonist as an active ingredient.

(2) The agent of the above-mentioned (1), wherein the promotion of gastrointestinal function is prophylaxis or improvement of a functional gastrointestinal disorder.

(3) The agent of the above-mentioned (2), wherein the functional gastrointestinal disorder is an upper gastrointestinal dysfunction.

(4) The agent of the above-mentioned (2), wherein the functional gastrointestinal disorder is functional dyspepsia or a gastroesophageal reflux disease.

(5) An appetite regulating agent, comprising a T1R agonist as an active ingredient.

(6) The agent of any one of the above-mentioned (1) to (5), wherein the T1R agonist is an amide derivative or Cyclamate.

(7) The agent of the above-mentioned (6), wherein the amide derivative is a compound represented by the following formula (I):

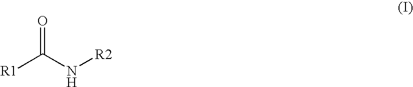

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,

R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),

or a pharmacologically acceptable salt thereof.

(8) The agent of the above-mentioned (6) or (7), wherein the amide derivative is a compound selected from the group consisting of:
(1) 3,6-dichloro-N-(4-ethoxyphenyl)-2-methoxybenzamide,
(2) 2,5-dichloro-N-(4-ethoxyphenyl)benzamide,
(3) N-(1-ethylpropyl)-benzofuran-2-carboxamide,
(4) N-(1,2,3,4-tetrahydronaphthalen-1-yl)-benzo[1,3]dioxol-5-carboxamide,
(5) 4-ethoxy-N-(1-propylbutyl)benzamide, and
(6) 3-(4-methoxyphenyl)-N-(1-propylbutyl)acrylamide.

(9) A pharmaceutical composition, comprising the agent of any one of the above-mentioned (1) to (8).

(10) A food or drink, comprising the agent of any one of the above-mentioned (1) to (8), wherein the active ingredient in said agent is contained in a proportion of 0.01 to 100,000 weight ppm of the food or drink.

(11) A method of screening for a substance capable of promoting a gastrointestinal function, which uses a cell expressing a T1R receptor.

(12) The method of the above-mentioned (11), wherein the substance capable of promoting a gastrointestinal function is a T1R agonist or T1R modulator.

(13) A method of screening for a substance capable of promoting a gastrointestinal function, which comprises the following steps (a), (b) and (c):

(a) contacting a test substance with a cell expressing a T1R receptor,

(b) determining the activation of G protein in the cell contacted with the test substance, and comparing the activation with that in a control cell free of a contact with the test substance, and

(c) selecting a substance capable of promoting a gastrointestinal function, based on the comparison results of the above-mentioned (b).

(14) The method of the above-mentioned (13), wherein an index for determining the activation of G protein is selected from an intracellular calcium concentration, an intracellular cAMP amount, an extracellular proton amount and an intracellular gastrointestinal hormone secretory amount.

(15) A method of screening for a substance capable of promoting a gastrointestinal function, which comprises the following steps (a), (b) and (c):

(a) contacting a test substance and a ligand acting on T1R receptor with a cell expressing a T1R receptor,

(b) measuring the amount of the ligand bound with a cell membrane of the cell, and comparing the amount with that in a control cell free of a contact with the test substance, and

(c) selecting a substance capable of promoting a gastrointestinal function, based on the comparison results of the above-mentioned (b).

(16) A method of promoting a gastrointestinal function, which comprises administering an effective amount of a T1R agonist to a mammal.

(17) The method of the above-mentioned (16), wherein the promotion of gastrointestinal function is prophylaxis or improvement of a functional gastrointestinal disorder.

(18) The method of the above-mentioned (17), wherein the aforementioned functional gastrointestinal disorder is an upper gastrointestinal dysfunction.

(19) The method of the above-mentioned (17), wherein the aforementioned functional gastrointestinal disorder is functional dyspepsia or a gastroesophageal reflux disease.

(20) A method of regulating an appetite, which comprises administering an effective amount of a T1R agonist to a mammal.

(21) The method of any one of the above-mentioned (16) to (20), wherein the T1R agonist is an amide derivative or Cyclamate.

(22) The method of the above-mentioned (21), wherein the amide derivative is a compound represented by the following formula (I):

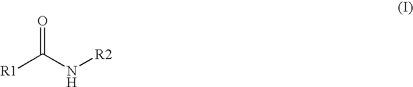

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,

R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),

or a pharmacologically acceptable salt thereof.

(23) The method of the above-mentioned (21) or (22), wherein the amide derivative is a compound selected from the group consisting of:
(1) 3,6-dichloro-N-(4-ethoxyphenyl)-2-methoxybenzamide,
(2) 2,5-dichloro-N-(4-ethoxyphenyl)benzamide,
(3) N-(1-ethylpropyl)-benzofuran-2-carboxamide,
(4) N-(1,2,3,4-tetrahydronaphthalen-1-yl)-benzo[1,3]dioxol-5-carboxamide,
(5) 4-ethoxy-N-(1-propylbutyl)benzamide, and
(6) 3-(4-methoxyphenyl)-N-(1-propylbutyl)acrylamide.

(24) The method of any one of the above-mentioned (16) to (23), which comprises administering a pharmaceutical composition comprising the aforementioned T1R agonist and a carrier to a mammal.

(25) The method of any one of the above-mentioned (16) to (23), which comprises administering a food or drink comprising the aforementioned T1R agonist in a proportion of 0.01 to 100,000 weight ppm to a mammal.

(26) Use of a T1R agonist for the production of a composition for promoting a gastrointestinal function.

(27) The use of the above-mentioned (26), wherein the promotion of gastrointestinal function is prophylaxis or improvement of a functional gastrointestinal disorder.

(28) The use of the above-mentioned (27), wherein the aforementioned functional gastrointestinal disorder is an upper gastrointestinal dysfunction.

(29) The use of the above-mentioned (27), wherein the aforementioned functional gastrointestinal disorder is functional dyspepsia or a gastroesophageal reflux disease.

(30) Use of a T1R agonist for the production of a composition for appetite regulation.

(31) The use of any one of the above-mentioned (26) to (30), wherein the T1R agonist is an amide derivative or Cyclamate.

(32) The use of the above-mentioned (31), wherein the amide derivative is a compound represented by the following formula (I):

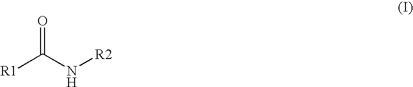

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,

R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),

or a pharmacologically acceptable salt thereof.

(33) The use of the above-mentioned (31) or (32), wherein the amide derivative is a compound selected from the group consisting of:
(1) 3,6-dichloro-N-(4-ethoxyphenyl)-2-methoxybenzamide,
(2) 2,5-dichloro-N-(4-ethoxyphenyl)benzamide,
(3) N-(1-ethylpropyl)-benzofuran-2-carboxamide,
(4) N-(1,2,3,4-tetrahydronaphthalen-1-yl)-benzo[1,3]dioxol-5-carboxamide,
(5) 4-ethoxy-N-(1-propylbutyl)benzamide, and
(6) 3-(4-methoxyphenyl)-N-(1-propylbutyl)acrylamide.

(34) The use of any one of the above-mentioned (26) to (33), wherein the aforementioned composition is a pharmaceutical product.

(35) The use of any one of the above-mentioned (26) to (33), wherein the aforementioned composition is a food or drink comprising a T1R agonist in a proportion of 0.01 to 100,000 weight ppm.

(36) A composition for promotion of a gastrointestinal function, comprising a T1R agonist as an active ingredient.

(37) The composition of the above-mentioned (36), wherein the promotion of gastrointestinal function is the prophylaxis improvement of a functional gastrointestinal disorder.

(38) The composition of the above-mentioned (37), wherein the aforementioned functional gastrointestinal disorder is an upper gastrointestinal dysfunction.

(39) The composition of the above-mentioned (37), wherein the aforementioned functional gastrointestinal disorder is functional dyspepsia or a gastroesophageal reflux disease.

(40) A composition for appetite regulation, comprising a T1R agonist as an active ingredient.

(41) The composition of any one of the above-mentioned (36) to (40), wherein the T1R agonist is an amide derivative or Cyclamate.

(42) The composition of the above-mentioned (41), wherein the amide derivative is a compound represented by the following formula (I):

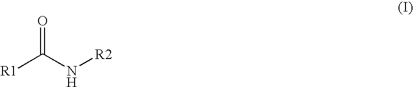

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,

R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),

or a pharmacologically acceptable salt thereof.

(43) The composition of the above-mentioned (41) or (42), wherein the amide derivative is a compound selected from the group consisting of:
(1) 3,6-dichloro-N-(4-ethoxyphenyl)-2-methoxybenzamide,
(2) 2,5-dichloro-N-(4-ethoxyphenyl)benzamide,
(3) N-(1-ethylpropyl)-benzofuran-2-carboxamide,
(4) N-(1,2,3,4-tetrahydronaphthalen-1-yl)-benzo[1,3]dioxol-5-carboxamide,
(5) 4-ethoxy-N-(1-propylbutyl)benzamide, and
(6) 3-(4-methoxyphenyl)-N-(1-propylbutyl)acrylamide.

(44) The composition of any one of the above-mentioned (36) to (43), which is a pharmaceutical product.

(45) The composition of any one of the above-mentioned (36) to (43), which is a food or drink comprising the active ingredient in a proportion of 0.01 to 100,000 weight ppm.

(46) A method of promoting a gastrointestinal function, which comprises administering an effective amount of at least one T1R agonist to a mammal in need thereof.

(47) The method of the above-mentioned (46), wherein said at least one T1R agonist is Cyclamate.

(48) The method of the above-mentioned (46), wherein said at least one T1R agonist is a compound represented by formula (I):

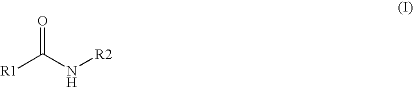

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,

R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),

or a pharmacologically acceptable salt thereof.

(49) The method of the above-mentioned (48), wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

(50) The method of the above-mentioned (48), wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

(51) The method of the above-mentioned (46), wherein said at least one T1R agonist is a compound represented by formula (II):

Ar1-Y-hAr1-X—(CR10R11)ₙ-hAr2 (II)

wherein Ar1 is an aryl group optionally having substituent(s) or a heteroaryl group optionally having substituent(s),

Y is single bond, O, S, S(O), SO₂, CR4R5 or NR6,

hAr1 is a heteroaryl group optionally having substituent(s),

X is O, S, SO, SO₂, CR7R8 or NR9,

n is an integer of 0 to 3,

hAr2 is an heteroaryl group optionally having substituent(s),

each of R4, R5, R7, R8, R10, and R11 is independently selected from hydrogen, oxygen, hydroxyl, NH₂, SH, halogen and C₁-C₄ organic group, and

each of R6 and R9 is independently selected from hydrogen, hydroxyl and C₁-C₄ organic group.

(52) The method of the above-mentioned (51), wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

(53) The method of the above-mentioned (46), which comprises administering a pharmaceutical composition comprising said at least one T1R agonist and a carrier to a mammal.

(54) The method of the above-mentioned (46), which comprises administering a food or drink comprising said at least one T1R agonist in an amount of 0.01 to 100,000 weight ppm to a mammal.

(55) A method of promoting a gastrointestinal function, which comprises administering an effective amount of at least one T1R agonist to a mammal in need thereof, wherein the promotion of gastrointestinal function is improvement of a functional gastrointestinal disorder.

(56) The method of the above-mentioned (55), wherein said at least one T1R agonist is Cyclamate.

(57) The method of the above-mentioned (55), wherein said at least one T1R agonist is a compound represented by formula (I):

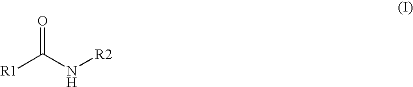

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,

R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),

or a pharmacologically acceptable salt thereof

(58) The method of the above-mentioned (55), wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

(59) The method of the above-mentioned (55), wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

(60) The method of the above-mentioned (55), wherein said at least one T1R agonist is a compound represented by formula (II):

Ar1-Y-hAr1-X—(CR10R11)ₙ-hAr2 (II)

wherein Ar1 is an aryl group optionally having substituent(s) or a heteroaryl group optionally having substituent(s),

Y is single bond, O, S, S(O), SO₂, CR4R5 or NR6,

hAr1 is a heteroaryl group optionally having substituent(s),

X is O, S, SO, SO₂, CR7R8 or NR9,

n is an integer of 0 to 3,

hAr2 is an heteroaryl group optionally having substituent(s),

each of R4, R5, R7, R8, R10, and R11 is independently selected from hydrogen, oxygen, hydroxyl, NH₂, SH, halogen and C₁-C₄ organic group, and

each of R6 and R9 is independently selected from hydrogen, hydroxyl and C₁-C₄ organic group.

(61) The method of the above-mentioned (55), wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

(62) The method of the above-mentioned (55), which comprises administering a pharmaceutical composition comprising said at least one T1R agonist and a carrier to a mammal.

(63) The method of the above-mentioned (55), which comprises administering a food or drink comprising said at least one T1R agonist in an amount of 0.01 to 100,000 weight ppm to a mammal.

(64) A method of promoting a gastrointestinal function, which comprises administering an effective amount of at least one T1R agonist to a mammal in need thereof, wherein the promotion of gastrointestinal function is improvement of an upper gastrointestinal dysfunction.

(65) The method of the above-mentioned (64), wherein said at least one T1R agonist is Cyclamate.

(66) The method of the above-mentioned (64), wherein said at least one T1R agonist is a compound represented by formula (I):

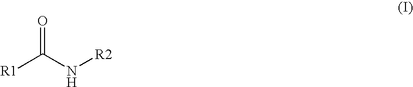

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,

R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),

or a pharmacologically acceptable salt thereof (67) The method of the above-mentioned (64), wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

(68) The method of the above-mentioned (64), wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

(69) The method of the above-mentioned (64), wherein said at least one T1R agonist is a compound represented by formula (II):

Ar1-Y-hAr1-X—(CR10R11)ₙ-hAr2 (II)
wherein Ar1 is an aryl group optionally having substituent(s) or a heteroaryl group optionally having substituent(s),

Y is single bond, O, S, S(O), SO₂, CR4R5 or NR6,

hAr1 is a heteroaryl group optionally having substituent(s),

X is O, S, SO, SO₂, CR7R8 or NR9,

n is an integer of 0 to 3,

hAr2 is an heteroaryl group optionally having substituent(s),

each of R4, R5, R7, R8, R10, and R11 is independently selected from hydrogen, oxygen, hydroxyl, NH₂, SH, halogen and C₁-C₄ organic group, and

each of R6 and R9 is independently selected from hydrogen, hydroxyl and C₁-C₄ organic group.

(70) The method of the above-mentioned (64), wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

(71) The method of the above-mentioned (64), which comprises administering a pharmaceutical composition comprising said at least one T1R agonist and a carrier to a mammal.

(72) The method of the above-mentioned (64), which comprises administering a food or drink comprising said at least one T1R agonist in an amount of 0.01 to 100,000 weight ppm to a mammal.

(73) A method of promoting a gastrointestinal function, which comprises administering an effective amount of at least one T1R agonist to a mammal in need thereof, wherein the promotion of gastrointestinal function is improvement of functional dyspepsia or a gastroesophageal reflux disease.

(74) The method of the above-mentioned (73), wherein said at least one T1R agonist is Cyclamate.

(75) The method of the above-mentioned (73), wherein said at least one T1R agonist is a compound represented by formula (I):

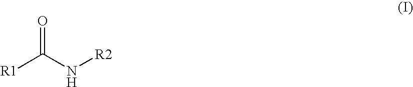

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,

R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),

or a pharmacologically acceptable salt thereof.

(76) The method of the above-mentioned (73), wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

(77) The method of the above-mentioned (73), wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

(78) The method of the above-mentioned (73), wherein said at least one T1R agonist is a compound represented by formula (II):

Ar1-Y-hAr1-X—(CR10R11)ₙ-hAr2 (II)

wherein Ar1 is an aryl group optionally having substituent(s) or a heteroaryl group optionally having substituent(s),

Y is single bond, O, S, S(O), SO₂, CR4R5 or NR6,

hAr1 is a heteroaryl group optionally having substituent(s),

X is O, S, SO, SO₂, CR7R8 or NR9,

n is an integer of 0 to 3,

hAr2 is an heteroaryl group optionally having substituent(s),

each of R4, R5, R7, R8, R10, and R11 is independently selected from hydrogen, oxygen, hydroxyl, NH₂, SH, halogen and C₁-C₄ organic group, and

each of R6 and R9 is independently selected from hydrogen, hydroxyl and C₁-C₄ organic group.

(79) The method of the above-mentioned (73), wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

(80) The method of the above-mentioned (73), which comprises administering a pharmaceutical composition comprising said at least one T1R agonist and a carrier to a mammal.

(81) The method of the above-mentioned (73), which comprises administering a food or drink comprising said at least one T1R agonist in an amount of 0.01 to 100,000 weight ppm to a mammal.

(82) A method of regulating an appetite, which comprises administering an effective amount of at least one T1R agonist to a mammal in need thereof.

(83) The method of the above-mentioned (82), wherein said at least one T1R agonist is Cyclamate.

(84) The method of the above-mentioned (82), wherein said at least one T1R agonist is a compound represented by formula (I):

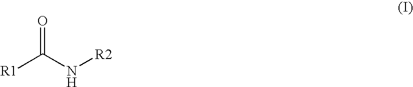

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,

R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),

or a pharmacologically acceptable salt thereof.

(85) The method of the above-mentioned (82), wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

(86) The method of the above-mentioned (82), wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

(87) The method of the above-mentioned (82), wherein said at least one T1R agonist is a compound represented by formula (II):

Ar1-Y-hAr1-X—(CR10R11)ₙ-hAr2 (II)

wherein Ar1 is an aryl group optionally having substituent(s) or a heteroaryl group optionally having substituent(s),

Y is single bond, O, S, S(O), SO₂, CR4R5 or NR6,

hAr1 is a heteroaryl group optionally having substituent(s),

X is O, S, SO, SO₂, CR7R8 or NR9,

n is an integer of 0 to 3,

hAr2 is an heteroaryl group optionally having substituent(s),

each of R4, R5, R7, R8, R10, and R11 is independently selected from hydrogen, oxygen, hydroxyl, NH₂, SH, halogen and C₁-C₄ organic group, and

each of R6 and R9 is independently selected from hydrogen, hydroxyl and C₁-C₄ organic group.

(88) The method of the above-mentioned (82), wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

(89) The method of the above-mentioned (82), which comprises administering a pharmaceutical composition comprising said at least one T1R agonist and a carrier to a mammal.

(90) The method of the above-mentioned (82), which comprises administering a food or drink comprising said at least one T1R agonist in an amount of 0.01 to 100,000 weight ppm to a mammal.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the results of immunostaining using an anti-T1R1 antibody, wherein the arrow shows stained cells, (A) is stomach.pyloric vestibular part, (B) is small intestine, and (C) is a taste cell of a taste bud.

FIG. 2 shows the results of double immunostaining in the same field using an anti-T1R1 antibody and an anti-gastrin antibody, wherein (A) is a T1R1 staining image, (B) is a gastrin staining image, and (C) is an overlap image of (A) and (B).

FIG. 3 shows the results of electrophoresis of a PCR product derived from each tissue, wherein the left end is an RNA marker, lanes 2-5 from the left end are amplification reaction products in the presence of a reverse transcriptase, and lanes 6-7 are reaction products in the absence of a reverse transcriptase (left end: RNA marker, lane 2: tongue.fungiform papillae, lane 3: tongue.non-taste bud tissue, lane 4: stomach.glandular stomach mucous membrane, lane 5: stomach.pyloric vestibular part mucous membrane).

FIG. 4 shows the gastric emptying rates when Cyclamate and MSG were administered.

FIG. 5 shows the gastric emptying rates when compound 1 (1 weight ppm and 10 weight ppm) was administered.

FIG. 6 shows the results of a gastrointestinal movement test where compounds 4, 5, and 6 (10 weight ppm, respectively) were administered.

FIG. 7 shows the gastric emptying rates when compounds 2, 3, 4, and 5 (10 weight ppm, respectively) were administered.

FIG. 8 shows the results of ¹³C-acetic acid breath test when compounds 7 to 15 were administered.

### DETAILED DESCRIPTION OF THE PREFERRED EMBODIMENTS

The present invention is explained in detail in the following.

In the present invention, the term “promotion of gastrointestinal function” means promotion of the motility of the gastrointestinal tract or promotion of digestion and absorption, which may be either a functional promotion by a direct action on the gastrointestinal tract, a secondary functional promotion via promotion of secretion (hormone etc.) in the endocrine system, improvement of blood flow and the like. For example, it includes improvement of various symptoms of the gastrointestinal tract showing degraded function due to gastrointestinal dysfunction, enhancement of gastrointestinal function of healthy individual, prophylaxis or improvement of disorders, prophylaxis or improvement of functional gastrointestinal disorders, and the like. Thus, the agent for the promotion of gastrointestinal function of the present invention and a composition containing the agent (composition for promoting gastrointestinal function) can be used for the promotion of gastrointestinal function, and can also be used as an agent for the prophylaxis or improvement of dyspepsia mentioned below, irrespective of the presence or absence of an organic disease.

As used herein, the term “functional gastrointestinal disorder” refers to a pathology where organic diseases such as peptic ulcer and cancer symptoms are not observed, but abdominal dyspepsia continues such as feeling of abdominal distention, nausea, vomiting, abdominal pain, anorexia, reflux of gastric acid, abnormal bowel movement (constipation, diarrhea and the like) and the like, based on the retention and the like of contents in gastrointestinal tract, particularly the stomach. It means a condition without organic disease of the gastrointestinal tract, but with a reproducible gastrointestinal symptom that degrades the QOL of patients. For example, it includes functional dyspepsia, gastroesophageal reflux disease, diabetic gastroparesis, reflux esophagitis, postoperative gastrointestinal dysfunction, gastroparesis, post-operative ileus, constipation, and the like. The term “gastrointestinal tract” in the present invention refers to a series of luminal organs involved in digestion from esophagus to anus and, for example, esophagus, stomach, small intestine (duodenum, jejunum, ileum) and large intestine can be mentioned.

The term “upper gastrointestin” refers to esophagus, stomach and duodenum, and the “upper gastrointestinal dysfunction” refers to the aforementioned dysfunction in the upper gastrointestine, and includes functional dyspepsia, diabetic gastroparesis, reflux esophagitis, postoperative gastrointestinal dysfunction, and the like.

As used herein, the term “functional dyspepsia” refers to a pathology where organic diseases such as peptic ulcer and cancer symptoms are not observed, but upper abdominal dyspepsia continues such as feeling of abdominal distention, nausea, vomiting, upper abdominal pain, anorexia, and the like, based on the retention and the like of the contents in the stomach and the like. It means a condition without organic disease of the gastrointestinal tract, but with a reproducible gastrointestinal symptom that degrades the QOL of patients. The dyspepsia includes diseases so far diagnosed as chronic gastritis and gastritis, and often shows symptoms of abdominal pain, heavy stomach, heartburn, and the like. In recent years, 40-60% of the outpatients of medical practitioners is said to suffer from functional dyspepsia, and Helicobacter pylori removal therapy tends to increase the number of functional dyspepsia.

Furthermore, the term “gastroesophageal reflux disease” includes reflux esophagitis and is developed by reflux of gastric acid and, in general, shows specific symptoms of heartburn, flow up of gastric acid to the mouth, and the like. Moreover, while “swallowing” means gulping water and food, it is closely related to not only mouth cavity and pharynx, but also motility of gastrointestinal tract such as esophagus and the like, as evidenced by misswallowing and vomiting due to sticking of a food bolus and the like in the esophagus and the like.

In the present invention, for example, the improvable specific symptoms of dyspepsia in the functional gastrointestinal disorders include, but not limited to, representative upper gastrointestinal dyspepsia such as nausea, vomiting, sickly feeling, heartburn, feeling of abdominal distention, heavy stomach, belching, chest writhing, chest pain, gastric discomfort, anorexia, dysphagia, reflux of gastric acid, and the like, lower gastrointestinal dyspepsia such as abdominal pain, constipation, diarrhea, and the like, and related complaints such as breathlessness, feeling of smothering, low incentive, pharyngeal obstruction.feeling of foreign substance (“baikakuki” in Chinese medicine), easy fatigability, stiff neck, myotonia, mouth dryness (dry mouth.thirst), tachypnea, burning sensation.cold sensation of extremities, difficulty in concentration, impatience, sleep disorder, headache, general malaise, palpitation, night sweat, anxiety, dizziness, vertigo, burning sensation, hot flash, sweating, abdominal pain, constipation, depression, and the like.

In the present invention, moreover, the term “appetite regulation” means enhancing the appetite of individual with anorexia, and leading individual with a tendency toward overeating to normal eating habits.

The gastrointestinal function promoter, the agent for the prophylaxis or improvement of functional gastrointestinal disorders and the appetite regulator of the present invention are used as agents for the prophylaxis or improvement of functional gastrointestinal disorders having reproducibility to degrade the QOL of patients, particularly upper gastrointestinal dysfunction such as functional dyspepsia, gastroesophageal reflux disease, and the like. They are hereinafter sometimes to be simply referred to as the agent of the present invention. In the present invention, the “improvement” or “improving” is meant to include “treatment” or “treating”.

The agent of the present invention contains a T1R agonist as an active ingredient. In the present invention, the term “T1R agonist” means a substance that enhances the activity of T1R receptor, which is a concept including not only a substance that binds with a T1R receptor to directly activate T1R receptor, but also a T1R modulator that expands the action of a T1R agonist. As the T1R agonist, various known T1R receptor agonists, and any compound that activates a T1R receptor may be used. Such compounds can be obtained by screening using a cell expressing a T1R receptor. Here, the T1R receptor means subunits of T1R1, T1R2 and T1R3, and any subunit or a combination of two or more subunits selected from these variants, and any agonist of any of these subunits or plural subunits can be used. T1R1, T1R2 and T1R3 may be a protein derived from a mammal such as human, monkey, mouse, dog, bovine and rabbit, or any animal such as bird, fish and the like, or may be a variant of these. The sequences of T1R1, T1R2 and T1R3 are each registered in the Gene bank as:

T1R1: mRNA Tas1r1, mouse NM_{—}031867, rat XM_{—}342986, human NM_{—}138697;
T1R2: mRNA Tas1R2, mouse NM_{—}031873, rat AF127390, human NM_{—}152232; and
T1R3: mRNA Tas1r3, mouse NM_{—}031872, rat NM_{—}130818, human XM_{—}371210.

As a known T1R agonist, Cyclamate (N-cyclohexylsulfamic acid) and, for example, compounds described in WO2005/041684, WO2006/084184, WO2006/084186, and WO2006/084246 can be mentioned.

The “T1R agonist” includes an amide derivative (compound having a partial structure of amide), specifically, for example, a compound having a partial structure of amide represented by the following formula (I) and pharmacologically acceptable salts thereof.

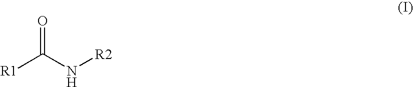

wherein:

R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—, where R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and

R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s).

The “T1R agonist” also includes a compound having a partial structure represented by the following formula (II) and pharmacologically acceptable salts thereof.

Ar1-Y-hAr1-X—(CR10R11)ₙ-hAr2 (II)

wherein:

Ar1 is an aryl group optionally having substituent(s) or a heteroaryl group optionally having substituent(s),

Y is single bond, O, S, S(O), SO₂, CR4R5 or NR6,

hAr1 is a heteroaryl group optionally having substituent(s),

X is O, S, SO, SO₂, CR7R8 or NR9,

n is an integer of 0 to 3,

hAr2 is an heteroaryl group optionally having substituent(s),

each of R4, R5, R7, R8, R10, and R11 is independently selected from hydrogen, oxygen, hydroxyl, NH₂, SH, halogen, and a C₁-C₄ organic group, and

R6 and R9 are independently selected from hydrogen, hydroxyl and a C₁-C₄ organic group.

Here, the “alkyl group having 2 to 25 carbon atoms” is a straight chain or branched chain alkyl group having 2 to 25, preferably 3 to 10, carbon atoms and, for example, methyl group, ethyl group, propyl group, isopropyl group, butyl group, isobutyl group, sec-butyl group, tert-butyl group, pentyl group, isopentyl group, neopentyl group, 1-methylbutyl group, 2-methylbutyl group, 2-ethylpropyl group, 1,1-dimethylpropyl group, 1,2-dimethylpropyl group, hexyl group, isohexyl group, 1-methylpentyl group, 2-methylpentyl group, 3-methylpentyl group, 1-ethylbutyl group, 2-ethylbutyl group, heptyl group, 1-methylhexyl group, 2-methylhexyl group, 3-methylhexyl group, 4-methylhexyl group, 5-methylhexyl group, 1-ethylpentyl group, 2-ethylpentyl group, 3-ethylpentyl group, 1-propylbutyl group, octyl group, nonyl group, decyl group, undecyl group, dodecyl group, tridecyl group, tetradecyl group, pentadecyl group, hexadecyl group, heptadecyl group, octadecyl group, nonadecyl group, icosyl group, henicosyl group, docosyl group, tricosyl group, tetracosyl group, pentacosyl group, and the like can be mentioned, with preference given to 1-ethylpropyl group, 1-propylbutyl group, and the like.

Here, the “cycloalkyl group having 3 to 25 carbon atoms” is a cycloalkyl group having 3 to 25, preferably 5 to 10, carbon atoms and, for example, cyclopropyl group, cyclobutyl group, cyclopentyl group, cyclohexyl group, cycloheptyl group, cyclooctyl group, cyclononyl group, cyclodecyl group, cycloundecyl group, cyclododecyl group, cyclotridecyl group, cyclotetradecyl group, cyclopentadecyl group, cyclohexadecyl group, cycloheptadecyl group, cyclooctadecyl group, cyclononadecyl group, cycloicosyl group, cyclohenicosyl group, cyclodocosyl group, cyclotricosyl group, cyclotetracosyl group, cyclopentacosyl group, and the like can be mentioned, with preference given to cyclohexyl group, cyclopentyl group, and the like.

The cycloalkyl group may be condensed with a benzene ring at any position. As the cycloalkyl group condensed with benzene, 1,2,3,4-tetrahydronaphthalen-1-yl, 1,2,3,4-tetrahydronaphthalen-2-yl, 2,3-dihydro-1H-inden-1-yl, and the like are preferable.

Here, the “aryl group” preferably has 6 to 14 carbon atoms, and is monocyclic or polycyclic aromatic hydrocarbon group. Specifically, for example, phenyl group, naphthyl group, and the like can be mentioned.

The aryl group may be condensed with a heterocyclic ring at any position. As the aryl group condensed with heterocyclic ring, 1,2,3,4-tetrahydroquinolinyl and the like are preferable.

Here, the “aralkyl group” is a group wherein one or more hydrogen atoms of alkyl group is/are substituted by aryl group, where the aryl group and alkyl group are as defined above. The alkyl moiety preferably has 1 to 3 carbon atoms. As concrete aralkyl groups, for example, benzyl group, phenylethyl group, 2-naphthylmethyl group, and the like can be mentioned.

Here, the “arylalkenyl group” is a group wherein one or more hydrogen atoms of alkenyl group is/are substituted by aryl group, where the aryl moiety to be contained is as defined for the above-mentioned aryl group. The alkenyl moiety preferably has 2 or 3 carbon atoms and, for example, vinyl, allyl, and the like can be mentioned. As the arylalkenyl group, for example, styryl group, cinnamyl group, and the like can be mentioned.

Here, the “heteroaryl group” means preferably 5- to 10-membered, monocyclic or polycyclic aromatic hetero ring groups, preferably containing, as ring atom(s), 1 to 4 hetero atoms selected from an oxygen atom, a sulfur atom and a nitrogen atom. Concretely, for example, as a 6-membered ring group, pyridyl group, pyridazinyl group, pyrimidyl group (=pyrimidinyl group) and pyrazinyl group can be mentioned; as a 5-membered ring group, furyl group, thienyl group, pyrrolyl group, isoxazolyl group, oxazolyl group, isothiazolyl group, thiazolyl group, pyrazolyl group, imidazolyl group, oxadiazolyl group, thiadiazolyl group, triazolyl group and tetrazolyl group can be mentioned; as a 6-5-membered ring group, benzofuranyl group, benzothienyl group, indolyl group, isoindolyl group, benzoxazolyl group (=benzooxazolyl group), benzothiazolyl group, benzimidazolyl group (=benzoimidazolyl group), indazolyl group, benzisoxazolyl group, benzisothiazolyl group, benzofurazanyl group, benzothiadiazolyl group, purinyl group and benzodioxolyl can be mentioned; as a 6-6-membered ring group, quinolyl group (=quinolinyl group), isoquinolyl group, cinnolinyl group, phthalazinyl group, quinazolinyl group, quinoxalinyl group, pteridinyl group can be mentioned; and as a 5-5-membered ring group, imidazooxazolyl group, imidazothiazolyl group, imidazoimidazolyl group, and the like can be mentioned.

Here, the “heteroaralkyl group” is a group wherein one or more hydrogen atoms of the alkyl group is/are substituted by a heteroaryl group, and the heteroaryl group and alkyl group to be contained are as defined above. The alkyl moiety preferably has 1 to 3 carbon atoms. Specifically, for example, 2-pyridylethyl group, benzofuranylmethyl group, and the like can be mentioned.

Here, the “heteroarylalkenyl group” is a group wherein one or more hydrogen atoms of the alkenyl group is/are substituted by a heteroaryl group, and the heteroaryl group and alkenyl group to be contained are as defined above. Specifically, for example, 2-pyridylethylene group and the like can be mentioned.

These groups may have one or more, preferably 1 to 3, substituents at substitutable positions. When two or more substituents are contained, the substituents may be the same or different. As the substituent, for example, halogen atom including fluorine, chlorine, bromine and iodine, hydroxyl group, oxo group, amino group, alkyl group having 1 to 6 carbon atoms such as methyl group, ethyl group, and the like, alkoxy group having 1 to 7 carbon atoms such as methoxy group, ethoxy group, methylenedioxy group, and the like, acyl group having 2 to 7 carbon atoms such as carboxyl group, acetyl group, propionyl group, and the like, alkoxycarbonyl group having 2 to 7 carbon atoms such as carbamoyl group, alkylcarbamoyl group having 2 to 10 carbon atoms, arylcarbamoyl group having 7 to 11 carbon atoms, heteroarylcarbamoyl group having 5 to 11 carbon atoms, arylalkylcarbamoyl group having 8 to 15 carbon atoms, heteroarylalkylcarbamoyl group having 6 to 15 carbon atoms, alkylthio group having 1 to 6 carbon atoms such as methylthio group, ethylthio group, and the like can be mentioned.

The substituent may further have a substituent such as the above-mentioned substituents at a substitutable position.

Here, the “C₁-C₄ organic group” means carbon-containing group, comprising 1 to 4 carbon atoms. The organic group can contain one or more heteroatoms, or be bonded to another molecule through a heteroatom, including oxygen, nitrogen, sulfur, phosphorus, or the like. Examples of organic group include but are not limited to alkyl or substituted alkyl, alkoxyl or substituted alkoxyl, hydroxyalkyl, alkoxyalkyl, cycloalkyl or substituted cycloalkyl, cycloalkenyl or substituted cycloalkyenyl, heterocycle or substituted heterocycle, aryl or substituted aryl, heteroaryl or substituted heteroaryl, mono or di-substituted amino, amide group, CN, CO₂H, CHO, COR, CO₂R, SR, wherein R is an alkyl, and the like. Examples of species of organic group include but are not limited to NHCH₃, N(CH₃)₂, CO₂CH₃, SEt, SCH₃, methyl, ethyl, isopropyl, vinyl, trifluoromethyl, methoxy, ethoxy, isopropoxy, trifluoromethoxy, phenyl, phenoxyl, and pyridyl groups, and the like.

As R1, “an aryl group optionally having substituent(s)”, “an arylalkenyl group optionally having substituent(s)”, “a heteroaryl group optionally having substituent(s)”, R3-NH—CO— and the like are preferable.

As the “aryl group optionally having substituent(s)” for R1, phenyl group optionally having substituents and the like are preferable. As the substituent, halogen atom, alkoxy group having 1 to 7, preferably 1 to 3, carbon atoms, and the like are preferable, chlorine, methoxy group, ethoxy group, methylenedioxy group and the like are particularly preferable.

As the “arylalkenyl group optionally having substituent(s)” for R1, styryl group optionally having substituents and the like are preferable. As the substituent, alkoxy group having 1 to 7, preferably 1 to 3, carbon atoms and the like are preferable, and methoxy group and the like are particularly preferable.

As the “heteroaryl group optionally having substituent(s)” for R1, benzofuranyl group optionally having substituents and the like are preferable.

As the “R3-NH—CO—” for R1, it is preferable when the R3 is an aralkyl group optionally having substituent(s) and the like. As the substituent, halogen atom, alkoxy group having 1 to 7, preferably 1 to 3, carbon atoms, and the like are preferable, chlorine, methoxy group, ethoxy group, methylenedioxy group and the like are particularly preferable.

Specifically, as R1, 3,6-dichloro-2-methoxyphenyl, 2,5-dichlorophenyl, 5-benzo[1,3]dioxol, 4-ethoxyphenyl, 2-(4-methoxyphenyl)vinyl, benzofuranyl, N-(2,4-dimethoxybenzyl)-aminocarbonyl, N-(2-chlorobenzyl)-aminocarbonyl, 2-amino-3-methoxyphenyl, 1,2,3,4-tetrahydroquinolin-7-yl, 3-chloro-4-methoxyphenyl, 4-methylthiophenyl, and the like are preferable.

As R2, “an aryl group optionally having substituent(s)”, “C₂₋₂₅ alkyl group optionally having substituent(s)”, “a C₃₋₂₅ cycloalkyl group optionally having substituent(s) (said cycloalkyl group is optionally condensed with benzene)”, “a heteroaralkyl group optionally having substituent(s)” and the like are preferable.

As the “aryl group optionally having substituent(s)” for R2, phenyl group optionally having substituent(s) and the like are preferable. As the substituent, alkoxy group having 1 to 7, preferably 1 to 3, carbon atoms and the like are preferable, and ethoxy group and the like are particularly preferable.

As the “C₂₋₂₅ alkyl group optionally having substituent(s)” for R2, a straight chain or branched chain alkyl group having 3 to 10 carbon atoms optionally having substituent(s) and the like are preferable, and a straight chain or branched chain alkyl group having 4 to 8 carbon atoms optionally having substituent(s) and the like are more preferable. As the substituent, alkyl group having 1 to 6 carbon atoms, alkoxycarbonyl group having 2 to 7 carbon atoms and the like are preferable, and methyl group, ethyl group, methoxycarbonyl group, ethoxycarbonyl group and the like are particularly preferable.

As the “a C₃₋₂₅ cycloalkyl group optionally having substituent(s)” for R2, cycloalkyl group condensed with a benzene ring optionally having substituent(s) and the like are preferable. As cycloalkyl group condensed with a benzene ring, 1,2,3,4-tetrahydronaphthalen-1-yl, 2,3-dihydro-1H-inden-1-yl, and the like are preferable. As the substituent, alkoxy group having 1 to 7, preferably 1 to 3, carbon atoms and the like are preferable, and methoxy group and the like are particularly preferable.

As the “a heteroaralkyl group optionally having substituent(s)” for R2, pyridylmethyl optionally having substituent(s), pyridylethyl optionally having substituent(s), pyridylpropyl optionally having substituent(s) and the like are preferable. As the substituent, alkyl group having 1 to 6, preferably 1 to 3, carbon atoms and the like are preferable, and methyl group and the like are particularly preferable.

Specifically, as R2, 4-ethoxyphenyl, 1-ethylpropyl, 1-propylbutyl, 1,2,3,4-tetrahydronaphthalen-1-yl, 2-(5-methylpyridin-2-yl)ethyl, 2-(pyridine-2-yl)ethyl, 5-methoxy-2,3-dihydro-1H-inden-1-yl, 6-methoxy-1,2,3,4-tetrahydronaphthalen-1-yl, 1-methoxycarbonyl-3-methyl-butan-1-yl, and the like are preferable.

As a compound represented by the formula (I), for example, the following compounds are preferable:
(1) 3,6-dichloro-N-(4-ethoxyphenyl)-2-methoxybenzamide,
(2) 2,5-dichloro-N-(4-ethoxyphenyl)benzamide,
(3) N-(1-ethylpropyl)-benzofuran-2-carboxamide,
(4) N-(1,2,3,4-tetrahydronaphthalen-1-yl)-benzo[1,3]dioxol-5-carboxamide,
(5) 4-ethoxy-N-(1-propylbutyl)benzamide,
(6) 3-(4-methoxyphenyl)-N-(1-propylbutyl)acrylamide,
(7) N-(2,4-dimethoxybenzyl)-N′-(2-(5-methylpyridin-2-yl)ethyl)oxalamide,
(8) N-(2-chlorobenzyl)-N′-(2-(pyridin-2-yl)ethyl)oxalamide,
(11) 2-amino-3-methoxy-N-(5-methoxy-2,3-dihydro-1H-inden-1-yl)benzamide,
(12) 2-amino-3-methoxy-N-(6-methoxy-1,2,3,4-tetrahydronaphthalen-1-yl)benzamide,
(13) N-(heptan-4-yl)-1,2,3,4-tetrahydroquinoline-7-carboxamide,
(14) (R)-methyl 2-(3-chloro-4-methoxybenzamido)-4-methylpentanoate, and
(15) (R)-methyl 4-methyl-2-(4-(methylthio)benzamido)pentanoate.

In the formula (II), as “Ar1”, an aryl group optionally having substituent(s) is preferable, a phenyl group optionally having substituent(s) is more preferable. As the substituent, alkyl group having 1 to 6, preferably 1 to 3, carbon atoms, alkoxy group having 1 to 7, preferably 1 to 3 and the like are preferable, and methyl group, methoxy group and the like are particularly preferable.

As “Y”, single bond is preferable.

As “hAr1”, 1,2,4-triazole optionally having substituent(s) is preferable.

As “X”, S is preferable.

As “n”, an integer of 1 is preferable.

As “hAr2”, is an heteroaryl group optionally having substituent(s) is preferable, a pyridyl group optionally having substituent(s) is more preferable.

As “R4”, “R5”, “R7”, “R8”, “R10” and “R11”, hydrogen is preferable.

As “R6” and “R9”, hydrogen is preferable.

As a compound represented by the formula (II), for example, the following compound is preferable:
(9) 2-((5-(2-methoxy-4-methylphenyl)-1H-1,2,4-triazol-3-ylthio)methyl)pyridine, and
(10) 2-((5-(2,4-dimethylphenyl)-1H-1,2,4-triazol-3-ylthio)methyl)pyridine.

The compounds represented by the formula (I) and formula (II) can be prepared by the procedure described in WO2005/041684, WO2006/084184, WO2006/084186, and WO2006/084246. Cyclamate is commercially available and can be prepared by the procedure described in U.S. Pat. No. 6,211,244, which is incorporated herein by reference in it entirety.

The T1R agonist to be used in the present invention, particularly, a compound represented by the formulae (I) and (II), may be in the form of a salt. As such salt, salts with an inorganic base, salts with an inorganic acid, salts with an organic acid, salts with an organic base, and the like can be mentioned, and it is not particularly limited as long as it is pharmacologically acceptable. As the salts with an inorganic base, alkali metal salts such as sodium, potassium, lithium, and the like, alkaline earth metal salts such as calcium, magnesium, and the like, ammonium salts and the like can be mentioned. As the salts with an inorganic acid, salts with hydrohalic acid (hydrochloric acid, hydrogen bromide acid, hydrogen iodide acid, etc.), sulfuric acid, nitric acid, phosphoric acid, and the like can be mentioned. As the salts with an organic acid, salts with formic acid, acetic acid, propionic acid, oxalic acid, succinic acid, maleic acid, fumaric acid, citric acid, glutamic acid, aspartic acid, histidine, and the like can be mentioned. As the salts with an organic base, salts with a basic amino acid (arginine, lysine, ornithine, and the like), nucleotide (purine derivative, pyrimidine derivative, and the like), alkaloid, and the like can be mentioned.

Furthermore, a substance (compound) that activates a T1R receptor from a known or novel compound obtained by the screening method of the present invention to be described in detail in the following, and the like may be used as a active ingredient of the present invention, which can promote a gastrointestinal function.

The screening method of the present invention is explained in the following.

The screening method of the present invention is characterized by screening “a substance capable of promoting a gastrointestinal function” by examining the presence or absence of activation of T1R receptor by a test substance using a cell expressing a T1R receptor. As the “substance capable of promoting a gastrointestinal function”, an agonist or modulator of T1R receptor can be mentioned, which is a substance that regulates the T1R receptor activity toward enhancement. The modulator of T1R receptor includes a substance that expands the activity of a T1R receptor agonist.

The presence or absence of activation of T1R receptor can be examined by measuring the amount of a substance binding therewith (ligand), a substance that inhibits the reaction of a signal that regulates the activity of T1R receptor, a substance (second messenger etc.) that transmits a signal produced by binding of a ligand with the T1R receptor, and the like. For example, activation of T1R receptor can be examined by detecting a second messenger produced by binding of a ligand such as glutamic acid and the like with the T1R receptor. In addition, activation of T1R receptor can also be detected by measuring the bond between a labeled ligand and a T1R receptor using a known labeled ligand.

Here, the T1R receptor acts on a GTP binding protein (also referred to as G protein: Gs, Gi, Gq, Ggust etc.) due to the binding of a ligand, and controls various cell functions via a second messenger such as cAMP and the like. Of these, intracellular calcium concentration increases by the activation of Gq. In addition, as the downstream increase of intracellular calcium concentration by signal transduction, activation of intracellular enzymes such as calmodulin, protein kinase C, adenylate cyclase and the like and functional regulation in the acute stage by phosphorylation of the cytoplasm.membrane protein can be mentioned. Activation of these intracellular enzymes changes channel function present in the cell membrane. It has also been found by the present inventors that T1R receptor expresses in gastrointestinal hormone-producing cells, particularly gastrin-producing cells. Therefore, the presence or absence of activation of T1R receptor by a test substance can be detected by contacting the test substance with a cell expressing a T1R receptor, and determining the activation of G protein using the measured value of intracellular calcium concentration, intracellular accumulation of cAMP, channel function (e.g., amount of extracellular proton production), gastrointestinal hormone secretion, and the like as an index.

The cell expressing a T1R receptor to be used in the screening method of the present invention may, for example, be a cell derived from a mammal such as mouse, rat, hamster, guinea pig, rabbit, dog, monkey, human and the like, a bird such as chicken and the like, and the like. Preferably, a gastrointestinal hormone-producing cell derived from the above-mentioned animal and the like are used.

A test substance for the screening method of the present invention may be any known compound or novel compound. For example, nucleic acid, carbohydrate, lipid, protein, peptide, an organic low-molecular-weight compound, a compound library constructed using a combinatorial chemistry technique, a random peptide library constructed using solid phase synthesis or a phage display method, or a natural component derived from a microorganism, plant or animal, a marine organism etc., and the like can be mentioned.

That is, the screening method of the present invention includes, for example, the following steps (a), (b) and (c):

(a) contacting a test substance with a cell expressing a T1R receptor,

(b) determining the activation of G protein in the cell contacted with the test substance, and comparing the activation with that in a control cell free of a contact with the test substance, and

(c) selecting a substance capable of promoting a gastrointestinal function, based on the comparison results of the above-mentioned (b).

In step (a) of the above-mentioned screening method (hereinafter to be also referred to as method A), a cell expressing a T1R receptor is placed in contact with a test substance. The test substance is contacted with the cell in a culture medium. The culture medium is appropriately selected according to the kind and the like of the cell to be used.

In step (b) of the above-mentioned screening method, the activation of G protein in a cell expressing a T1R receptor is first evaluated in the presence of a test substance. Then, the activation is compared with the activation in the absence of the test substance. As the index for determining the activation of G protein, an intracellular calcium concentration, an intracellular cAMP amount, an extracellular proton amount, an intracellular gastrointestinal hormone secretion amount and the like can be mentioned.

In step (c) of the above-mentioned screening method, the activation is compared, for example, based on the presence or absence of a significant difference. As a result of the evaluation, when an increase.expansion of the activation can be confirmed, in the presence of the test substance relative to the absence thereof, the test substance can be judged to be a substance capable of promoting a gastrointestinal function.

When a T1R modulator is screened for, it is possible that a test substance and a T1R agonist are contacted with a cell expressing a T1R receptor in the above-mentioned step (a), activation of G protein when a T1R agonist is contacted with the cell in the presence of a test substance and activation of G protein when a T1R agonist is contacted with the cell in the absence of a test substance are compared in (b), and the substance that expanded the activation of G protein is selected as a substance capable of promoting a gastrointestinal function (T1R modulator) in (c).

Moreover, another screening method of the present invention includes, for example, the following steps (a), (b) and (c):

(a) contacting a test substance and a ligand acting on T1R receptor with a cell expressing a T1R receptor,

(b) measuring the amount of the ligand bound with a cell membrane of the cell, and comparing the amount with that in a control cell free of a contact with the test substance,

(c) selecting a substance capable of promoting a gastrointestinal function, based on the comparison results of the above-mentioned (b).

In step (a) of the above-mentioned screening method, a cell expressing a T1R receptor is placed in contact with a test substance and a ligand acting on T1R receptor. The test substance and a ligand acting on T1R receptor are contacted with the cell in a culture medium. The culture medium is appropriately selected according to the kind and the like of the cell to be used.

In step (b) of the above-mentioned screening method, the amount of the ligand bound with the cell membrane of a cell expressing a T1R receptor is first evaluated in the presence of a test substance. Then, the amount of the ligand is compared with that in the absence of the test substance. The amount of the bound ligand can be, for example, measured using radiolabeled ligand and the like.

In step (c) of the above-mentioned screening method, the amount of the ligand is compared, for example, based on the presence or absence of a significant difference. As a result of the evaluation, when a decrease in the amount of the ligand bound can be confirmed in the presence of the test substance relative to the absence thereof, the test substance can be judged to be a substance capable of promoting a gastrointestinal function.

Furthermore, a substance wherein a decrease in the ligand binding amount could be confirmed can be confirmed as a T1R agonist by the aforementioned screening method A.

While the ligand acting on T1R is not particularly limited, for example, glutamic acid, nucleic acid, and the like can be mentioned.

Specific methods (1)-(6) for detecting a substance capable of promoting a gastrointestinal function are shown below, which use a cell expressing a T1R receptor (cell functionally retaining a T1R receptor).

(1) A method comprising the following step (a), (b) and (c):

(a) contacting a test substance with a cell expressing a T1R receptor, into which a calcium sensitive dye (e.g., Fura-2, Indo-1, Fluo-3 etc.) has been introduced, for a given period,

(b) determining the fluorescence intensity (intracellular calcium concentration) in the cell contacted with the test substance, and comparing the intensity with that of a control cell free of a contact with the test substance, and

(c) selecting a substance capable of promoting a gastrointestinal function, based on the comparison results of the above-mentioned (b).

In step (a) of the above-mentioned screening method, the cell expressing a T1R receptor, which is contacted with the test substance, is preferably a gastrointestinal hormone-producing cell that expresses a T1R receptor. For example, the object substance may be searched for based on the changes in the fluorescence intensity (intracellular calcium concentration) when a test substance is contacted with a gastrointestinal hormone-producing cell, into which a calcium sensitive dye has been introduced, for a given period. When a T1R modulator is screened for, a test substance and a T1R agonist may be contacted with a cell expressing a T1R receptor into which a calcium sensitive dye (e.g., Fura-2, Indo-1, Fluo-3 etc.) has been introduced.

In step (b) of the above-mentioned screening method, whether or not the fluorescence intensity (intracellular calcium concentration) of a cell expressing a T1R receptor in the presence of a test substance changes is evaluated. That is, evaluation is made by comparing the measured fluorescence intensity (intracellular calcium concentration) with that in the absence of a test substance. The fluorescence intensity can be measured by a method known per se. When a T1R modulator is to be screened for, the fluorescence intensity when a T1R agonist is contacted with a cell expressing a T1R receptor in the presence of a test substance may be compared with that when a T1R agonist is contacted with the cell in the absence of the test substance.

In step (c) of the above-mentioned screening method, the fluorescence intensity is compared, for example, based on the presence or absence of a significant difference. As a result of the evaluation of the fluorescence intensity, when an increase in the intracellular calcium concentration can be confirmed, the test substance can be judged to be a substance capable of promoting a gastrointestinal function. When a T1R modulator is to be screened for, a substance that expanded the range of fluorescence intensity shift may be selected as a substance capable of promoting a gastrointestinal function (T1R modulator).

(2) A method comprising the following steps (a), (b) and (c):

(a) contacting a test substance with a cell expressing a T1R receptor for a given period,

(b) measuring the cAMP amount in the cell contacted with the test substance, and comparing to that in a control cell free of a contact with the test substance, and

(c) selecting a substance capable of promoting a gastrointestinal function, based on the comparison results of the above-mentioned (b).

The above-mentioned steps (a) and (b) can be performed, for example, based on the description of Chaudhari N, Nat Neurosci, 2000 February; 3(2): 113-9; Flor P J, Neuropharmacology, 1995 February; 34(2): 149-55.

The cAMP amount can be measured using a commercially available assay kit.

In step (a) of the above-mentioned screening method, when a T1R modulator is to be screened for, a test substance and a T1R agonist may be contacted with a cell expressing a T1R receptor.

In step (b) of the above-mentioned screening method, when a T1R modulator is to be screened for, the cAMP amount when a T1R agonist is contacted with a cell expressing a T1R receptor in the presence of a test substance may be compared with that when a T1R agonist is contacted with the cell in the absence of the test substance.

In step (c) of the above-mentioned screening method, the cAMP amount is compared, for example, based on the presence or absence of a significant difference. As a result of the evaluation of the cAMP amount, when an increase in the cAMP amount can be confirmed, the test substance can be judged to be a substance capable of promoting a gastrointestinal function. When a T1R modulator is to be screened for, a substance that enhanced an increase in the cAMP amount may be selected as a substance capable of promoting a gastrointestinal function (T1R modulator).

(3) A method comprising the following steps (a), (b) and (c):

(a) contacting a test substance and a known ligand (e.g., glutamic acid, nucleic acid etc.) acting on T1R receptor with a cell expressing a T1R receptor for a given period,

(b) measuring the amount of the ligand bound with a cell membrane of the cell, and comparing the amount with that in a control cell free of a contact with the test substance,

(c) selecting a substance capable of promoting a gastrointestinal function, based on the comparison results of the above-mentioned (b).

The above-mentioned steps (a) and (b) can be performed, for example, based on the descriptions of Naples M A, Neuropharmacology, 2001; 40(2): 170-7; Thomsen C, Neuropharmacology, 1997 January; 36(1): 21-30.

The amount of a known ligand can be measured by radioactively labeling a part of the substance and measuring the amount of the radioactivity bound with the cell membrane.

In step (c) of the above-mentioned screening method, the amount of the ligand is compared, for example, based on the presence or absence of a significant difference. As a result of the evaluation of the amount of the ligand, when an increase in the amount of the ligand bound can be confirmed, the test substance can be judged to be a substance capable of promoting a gastrointestinal function.

(4) A method comprising the following steps (a), (b) and (c):

(a) contacting a test substance with a cell expressing a T1R receptor, into which a cAMP sensitive fluorescent protein (e.g., F1CRhR etc.) has been introduced, for a given period,

(b) determining the fluorescence intensity (intracellular cAMP concentration) in the cell contacted with the test substance, and comparing that in a control cell free of a contact with the test substance, and

(c) selecting a substance capable of promoting a gastrointestinal function, based on the comparison results of the above-mentioned (b).

The above-mentioned steps (a) and (b) can be performed, for example, based on Adams S R, Nature, 1991 Feb. 21; 349(6311): 694-7.

Here, the cell expressing a T1R receptor is preferably a gastrointestinal hormone-producing cell expressing the T1R receptor.

In step (a) of the above-mentioned screening method, when a T1R modulator is to be screened for, a test substance and a T1R agonist may be contacted with a cell expressing a T1R receptor, into which a cAMP sensitive fluorescent protein (e.g., F1CRhR etc.) has been introduced.

In step (b) of the above-mentioned screening method, when a T1R modulator is to be screened for, the fluorescence intensity (intracellular cAMP concentration) when a T1R agonist is contacted with a cell in the presence of a test substance may be compared with that when a T1R agonist is contacted with the cell in the absence of the test substance.

In step (c) of the above-mentioned screening method, the fluorescence intensity is compared, for example, based on the presence or absence of a significant difference. As a result of the evaluation of the fluorescence intensity, when an increase in the fluorescence intensity can be confirmed, the test substance can be judged to be a substance capable of promoting a gastrointestinal function. When a T1R modulator is to be screened for, a substance that enhanced the increase of fluorescence intensity may be selected as a substance capable of promoting a gastrointestinal function (T1R modulator).

(5) A method comprising the following steps (a), (b) and (c):

(a) contacting a test substance with a cell expressing a T1R receptor for a given period,

(b) measuring the amount of extracellular proton production in the cell contacted with the test substance, and comparing the proton production amount with that in a control cell free of a contact with the test substance, and

(c) selecting a substance capable of promoting a gastrointestinal function, based on the comparison results of the above-mentioned (b).

The above-mentioned steps (a), (b) and (c) can be performed based on, for example, the description of McConnell H M, Science, 1992 Sep. 25; 257(5078): 1906-12.

Here, the cell expressing a T1R receptor is preferably a gastrointestinal hormone producing cell that expresses a T1R receptor and, for example, the extracellular proton production amount when a T1R receptor agonist and a test substance are contacted with a gastrointestinal hormone cell that expresses the T1R receptor for a given period is measured, and the object substance may be detected using the proton production amount as an index. The amount of proton production is measured by a site sensor.

In step (a) of the above-mentioned screening method, when a T1R modulator is to be screened for, a test substance and a T1R agonist may be contacted with a cell expressing a T1R receptor.

In step (b) of the above-mentioned screening method, when a T1R modulator is to be screened for, the amount of extracellular proton production when a T1R agonist is contacted with a cell expressing a T1R receptor in the presence of a test substance may be compared with that when a T1R agonist is contacted with the cell in the absence of the test substance.

In step (c) of the above-mentioned screening method, the proton production amount is compared, for example, based on the presence or absence of a significant difference. As a result of the evaluation of the proton production amount, when an increase in the extracellular proton production amount can be confirmed, the test substance can be judged to be a substance capable of promoting a gastrointestinal function. When a T1R modulator is to be screened for, a substance that enhanced an increase in the extracellular proton production amount may be selected as a substance capable of promoting a gastrointestinal function (T1R modulator).

(6) A method comprising the following steps (a), (b) and (c):

(a) contacting a test substance with a cell expressing a T1R receptor for a given period,

(b) measuring the amount of gastrointestinal hormone secretion in the cell contacted with the test substance, and comparing the amount of gastrointestinal hormone secretion with that of a control cell free of a contact with the test substance, and

(c) selecting a substance capable of promoting a gastrointestinal function, based on the comparison results of the above-mentioned (b).

Here, the cell expressing a T1R receptor is preferably a gastrointestinal hormone producing cell that expresses a T1R receptor and, for example, the amount of gastrointestinal hormone secretion when a T1R receptor agonist and a test substance are contacted with a gastrointestinal hormone producing cell expressing a T1R receptor for a given period is measured, and the object substance may be searched for using the amount of gastrointestinal hormone secretion as an index.

The amount of gastrointestinal hormone secretion can be measured using a commercially available assay kit.

In step (a) of the above-mentioned screening method, when a T1R modulator is to be screened for, a test substance and a T1R agonist may be contacted with a cell expressing a T1R receptor.

In step (b) of the above-mentioned screening method, when a T1R modulator is to be screened for, the gastrointestinal hormone secretion amount when a T1R agonist is contacted with a cell expressing a T1R receptor in the presence of a test substance may be compared with that when a T1R agonist is contacted with the cell in the absence of the test substance.

In step (c) of the above-mentioned screening method, the amount of gastrointestinal hormone secretion is compared, for example, based on the presence or absence of a significant difference. As a result of the evaluation of the gastrointestinal hormone secretion amount, when the variation of the gastrointestinal hormone secretion amount can be confirmed, the test substance can be judged to be a substance capable of promoting a gastrointestinal function. When a T1R modulator is to be screened for, a substance that expanded the range of shift of the amount of gastrointestinal hormone secretion may be selected as a substance capable of promoting a gastrointestinal function (T1R modulator).

The agent of the present invention is useful as a pharmaceutical agent, food and drink and the like, and the subject of administration is, for example, mammals (e.g., human, mouse, rat, hamster, rabbit, cat, dog, bovine, sheep, monkey etc.) and the like. According to the present invention, moreover, use of a T1R agonist for the production of a composition for promoting gastrointestinal function and controlling appetite, and a method of promoting gastrointestinal function and controlling appetite, which comprises administering an effective amount of a T1R agonist to a mammal, are provided.

When the agent of the present invention is contained in a pharmaceutical composition, the pharmaceutical composition generally contains a T1R agonist and a carrier. While the carrier is not particularly limited as long as it is acceptable as a pharmaceutical agent and, for example, the below-mentioned substances (e.g., excipient, solvent etc.) for preparation can be mentioned.

As used herein, while the administration mode of the agent or pharmaceutical composition of the present invention (hereinafter also to be simply referred to as a pharmaceutical agent) is not particularly limited, general administration routes such as oral administration, rectal administration, administration by injection or transfusion, and the like can be employed.

The dosage form for oral administration includes granule, fine granule, powder, coated tablet, tablet, suppository, powder, (micro)capsule, chewable, syrup, juice, liquid, suspension, emulsion, and the like. For injection, general dosage forms of pharmaceutical preparations such as direct intravenous injection, drip infusion, preparation prolonging the release of activity substance and the like can be employed.

These pharmaceutical agents can be formulated according to a conventional method. When necessary for formulation, pharmacologically acceptable various substances (as auxiliaries) for preparations can be added. While the substance for preparation can be appropriately selected according to the dosage form of the preparation, it includes, for example, excipient, diluent, additive, disintegrant, binder, coating agent, lubricant, glidant, lubricant, flavor, sweetener, solubilizer, solvent and the like. Specific examples of the substance for preparation include magnesium carbonate, titanium dioxide, lactose, mannitol and other saccharides, talc, milk protein, gelatin, starch, cellulose and derivatives thereof, animal and vegetable oil, polyethylene glycol, and a solvent, such as sterile water and monovalent or polyvalent alcohol (e.g., glycerol and the like).

While the dose of the pharmaceutical agent of the present invention for oral administration varies depending on the symptoms and age of the patients to be the subjects of administration, and administration method, the daily dose of the active ingredient for an adult (body weight 60 kg) is generally about 0.001 mg to 1 g, preferably about 0.01 mg to 1 g, and more preferably about 0.1 mg to 1 g.

The dose of parenteral administration (intake) by way of drip infusion, injection (transvenous administration) and the like is about 1/10 to 1/20 of the aforementioned preferable dose (intake amount) by oral administration.

The pharmaceutical agent of the present invention may be used in combination with other pharmaceutical agents, and as such pharmaceutical agents, for example, acid secretion inhibitors such as H2 receptor antagonists, proton pump inhibitors, and the like, motility function improvers such as 5-HT receptor agonist, D2 antagonist, and the like, antacid agents such as muscarine receptor antagonists, anti-gastrin drugs, anticholinergic drugs, and the like, mucous membrane protectors such as teprenone, plaunotol, omoprostil, enprostil, misoprostol, rebamipide, sucralfate, polaprezinc, azulene, egualen sodium, glutamine, aldioxa, gefamate, ecabet sodium, and the like, inflammatory colitis treating agents such as sulfasalazine, 5-ASA preparation, steroids, remicade, and the like can be used. One or more kinds of these can be contained.

The agent of the present invention may be contained in food or drink. When contained in food or drink, any conventional diet form can be employed as long as it contains the active ingredient of the present invention. For example, a suitable flavor may be added to give a drink, such as refreshing beverage and powder beverage. Specifically, for example, it can be mixed with juice, milk, confectionery, jelly and the like and served. It is also possible to provide such food and drink as a Food with Health Claims as defined by the Ministry of Health, Labour and Welfare, which includes food and drink, particularly Food for Specified Health Uses, Food with Nutrient Function Claims and the like, indicating use of the present invention for promotion of gastrointestinal function and appetite regulation, and the like.

Moreover, it is also possible to add the agent of the present invention to a condensed fluid diet or use same as a dietary supplement. For use as a dietary supplement, for example, it can be formed into tablet, capsule, powder, granule, suspension, chewable, syrup and the like. The dietary supplement in the present invention includes, in addition to those taken as food, those taken for the purpose of supplementing nutrition, which include nutritional supplement, supplement (particularly dietary supplement) and the like.

When the agent of the present invention is contained in a food or drink, the amount of intake of the active ingredient for an adult per day is generally about 0.001 mg to 1 g, preferably about 0.01 mg to 1 g, and more preferably about 0.1 mg to 1 g. When the agent of the present invention is contained in a food or drink, the content of the active ingredient in the food or drink is generally about 0.01 to 100,000 weight ppm, preferably about 0.01 to 10,000 weight ppm, and more preferably about 0.01 to 1000 weight ppm.

Other features of the invention will become apparent in the course of the following descriptions of exemplary embodiments which are given for illustration of the invention and are not intended to be limiting thereof.

### EXAMPLES

### Example 1

### Identification of Locality of T1R1 Receptor by Immunostaining

### (1) Preparation of Sectional Specimen of Rat Stomach and Small Intestine.

Rat (Sprague-Dawley, male, 350 to 400 g) was sacrificed by exsanguinations after incising the heart right auricle under etherization, and the stomach and small intestine were recovered immediately thereafter. From the stomach, the pyloric vestibular part was recovered where many gastrointestinal hormone-producing cells are distributed, and from the small intestine, a part about 5 cm from the stomach pyloric was recovered. When a large amount of the digest was left in the gastrointestine, the intestine was washed with saline.

The removed stomach and the intestine were incised, pinned on a corkboard, and shaken in 4% para-formaldehyde (4° C.) for one day for immersion fixation. Thereafter, they were cryoprotected by immersion in 20% Sucrose-PBS for 3-4 days, embedded in an embedding agent (OCT compound, trade name: Tissue-Tek, Sakura Seiki Co., Ltd.) and sliced in 5 to 7 μm with cyrostat. The section was dried at room temperature and preserved at 4° C. until use for various stainings.

### (2) Immunostaining Using Anti-T1R1 Receptor Antibody.

The section was immunostained according to the method described in a known publication (Drengk et al., J. Auto. Nerv. Sys., 78: 109-112, 2000; Miampamba & Sharkey, J. Auto. Nerv. Sys., 77: 140-151, 1999).

The section was washed with PBS, and treated with 3% hydrogen peroxide methanol for 15 minutes to prevent reaction by endogenous peroxidase. Then, the section was washed with PBS and blocked for 1 hour using 1% bovine serum albumin added PBS (1% BSA-PBS) containing 10% normal horse serum. The section was washed with PBS again, and reacted with a primary antibody (Table 1) diluted with 1% BSA-PBS containing 1% normal horse serum at 4° C. for 2 nights. Then, the section was washed with PBS, and reacted with a secondary antibody (Table 1) diluted with 1% BSA-PBS at room temperature for 1 hour. Finally, an ABC (Avidin-biotin complex) reaction was carried out using a Vectorstain elite kit (Vector) and treated with 0.025% diaminobenzidine to allow color development. After completion of the reaction, the section was washed with PBS, dehydrated with ethanolexylene, included and observed with a microscope. A section free of a primary antibody was used as a negative control. The kind and dilution rate of the primary and secondary antibodies used are shown in Table 1.

TABLE 1

| | primary | | secondary |
|---|---|---|---|
| | antibody | | antibody |
| | dilution | | dilution |
| primary antibody | rate | secondary antibody | rate |
| anti-rat T1R1, rabbit, | 100 | biotinylated goat anti- | 500 |
| polyclonal, | | rabbit IgG (Jackson | |
| Alpha Diagnostic | | ImmunoResarch, West | |
| International, USA cat# | | Grove, PA) | |
| TR11-A | | | |

### (3) Hematoxylin Staining.

A section was washed with water, nuclear stained with Mayer's hematoxylin (Wako Pure Chemical Industries, Ltd.) and, after color development, applied to dehydration.inclusion.

### (4) Results.

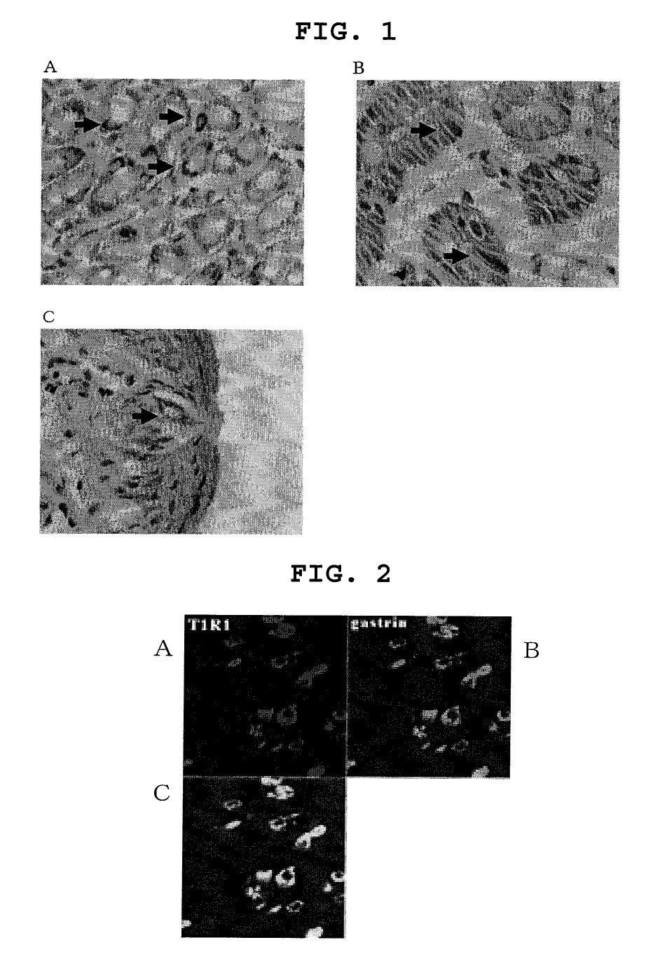

The results of immunostaining are shown in FIG. 1. In the stomach (FIG. 1A) and small intestine (FIG. 1B), the cells scattered in the gastrointestinal mucous membrane were stained due to anti-T1R1 receptor antibody. From the morphological characteristic in that the upper end of the positive cell faces the lumen of the intestine in both stomach and small intestine, the cells were assumed to be gastrointestinal hormone-producing cells. Heretofore, the expression of T1R1 receptor in gastrointestinal hormone-producing cells is not known. Since T1R1 receptor expressed in the gastrointestinal hormone-producing cells, functional relationship with endocrine regulation of gastrointestinal hormone was suggested. It was confirmed that anti-T1R1 receptor antibody stained the taste cell of a taste bud (FIG. 1C).

### Example 2

### Identification of Locality of T1R1 Receptor and Gastrin by Double Immunostaining

### (1) Double Staining by Anti-T1R1 Receptor Antibody and Anti-Gastrin Antibody.

A stomach section was subjected to double staining by anti-T1R1 receptor antibody and anti-gastrin antibody. A section was first washed with PBS, and blocked using 1% bovine serum albumin-added PBS (1% BSA-PBS) containing 10% normal horse serum for 1 hour. The section was washed again with PBS, and a mixture (Table 2) of primary antibody diluted with 1% BSA-PBS containing 1% normal horse serum was reacted at 4° C. for two nights. Then, the section was washed with PBS, and reacted with a secondary antibody (Table 2) diluted with 1% BSA-PBS at room temperature for 2 hour. After completion of the reaction, the section was washed with PBS, dehydrated with ethanol.exylene, included and observed with a confocul laser microscope (LSM 510; Zeiss, Germany). A section free of a primary antibody was used as a negative control. The kind and dilution rate of the primary and secondary antibodies used are shown in Table 2.

TABLE 2

| | primary | | primary |
|---|---|---|---|
| primary antibody mixture | antibody | secondary antibody | antibody |
| composition | dilution | mixture composition | dilution |
| (1) + (2) | rate | (3) + (4) | rate |
| (1) anti-rat T1R1, rabbit, | 100 | (3) Cy3-lated anti- | 100 |
| polyclonal, | | rabbit IgG | |
| (Alpha Diagnostic | | | |
| International, USA cat# | | | |
| TR11-A) | | | |
| (2) anti-gastrin, goat, | 200 | (4) Alexa Fluoro 488- | 100 |
| polyclonal | or | labeled anti-goat IgG | |
| (Santa Cruz | 400 | | |
| Biotechnology Inc., CA) | | | |

### (2) Results.

The results of immunostaining observed in the same field by a confocal microscope are shown in FIG. 2. The cells scattered in the stomach mucous membrane were stained by anti-T1R1 receptor antibody (FIG. 2A) and anti-gastrin antibody (FIG. 2B). In a superimposed picture (FIG. 2C) of FIG. 2A and FIG. 2B, the stained images matched with each other. Therefrom it was clarified that the T1R1 positive cells in the stomach were gastrin (one of the gastrointestinal hormones)-producing cells. Since T1R1 was expressed in gastrin producing cells, a functional relationship with gastrin endocrine regulation was suggested.

### Example 3

### Detection of T1R1 mRNA Expression in Stomach by Molecule Biological Method

(1) Amplification of T1R1 mRNA Partial Sequence.

The mucous membranes of glandular stomach and pyloric vestibular part were taken from the stomach of rat (Sprague-Dawley). From the tongue, fungiform papillae including a taste bud and a tissue without a taste bud were taken. Using total RNA extracted from the sample of each part of the stomach and tongue as a template and a SuperScrip reverse transcriptase enzyme (Invitrogen, CA, USA), reverse transcription was performed. Using the obtained cDNA as a template, T1R1 was amplified using the following gene-specific primer and LA taq (TaKaRa).

The gene specific primers used are shown below.

SEQ ID NO: 1:
T1R1-824 Forward 5′-AGGACCACCGTGGTCGTGGTCTT-3′
SEQ ID NO: 2:
T1R1-2163 Reverse 5′-GCACTCAAGAATCACCAGATGGG-3′

### (2) Results.

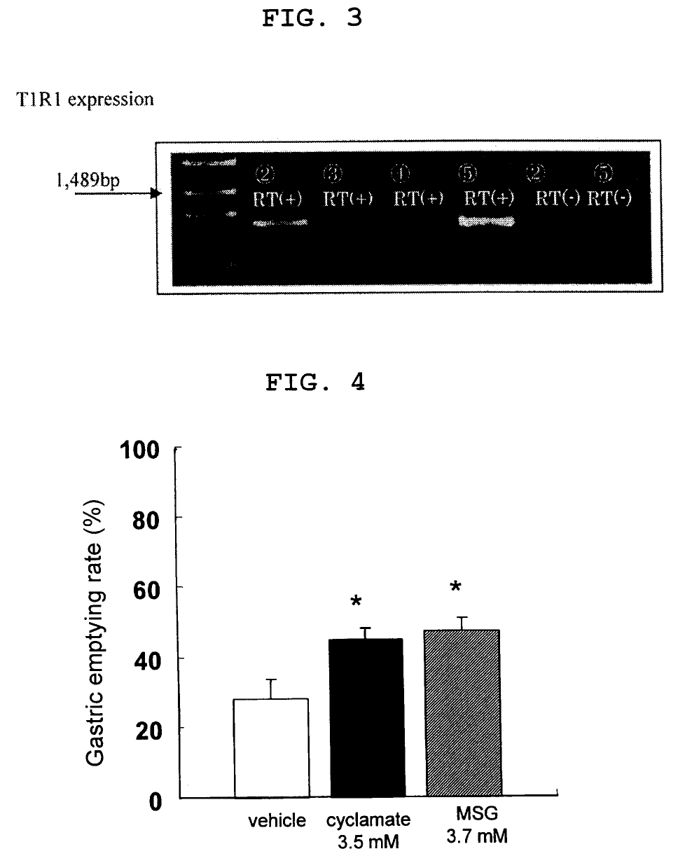

The same size of PCR products were obtained from the samples derived from stomach.pyloric vestibular part mucous membrane (FIG. 3, line 5) and tongue.fungiform papillae (FIG. 3, line 2). Sequence analysis revealed that these PCR products have the same sequence as that of T1R1 derived from taste cell. On the other hand, PCR products were not obtained from the stomach.glandular stomach mucous membranes (FIG. 3, line 4) and tongue.non-taste bud tissue (FIG. 3, line 3). In addition, PCR product was not obtained from the samples (FIG. 3, lines 6 and 7) that underwent an amplification reaction operation without reverse transcriptase. The stomach.pyloric vestibular part is particularly known as a part where gastrin producing cells are distributed. By this Example, expression of T1R1 in the mucous membrane of the stomach.pyloric vestibular part was established not only by an immunity tissue chemical method but also by a molecule biological method.

### Example 4

### Stomach Content Emptying Test Using T1R Agonist

### Experiment Method.

### Mouse Gastric Emptying Method.

Male ICR mouse was used. A 5% casein fluid diet (0.5 mL) containing 0.05% phenol red and a test drug (3.5 mM Cyclamate, 3.7 mM MSG (monosodium glutamate), compound 1 (1 weight ppm and 10 weight ppm) of the following Production Example 1) was orally administered, and 30 minutes later, the chest was opened and the stomach was isolated. The stomach was placed in 0.1N sodium hydroxide (14 mL), homogenized and left standing for 1 hour at room temperature. 20% Trichloroacetic acid (0.5 mL) was added to 5 mL of the supernatant, and the mixture was centrifuged (3000 rpm, 20 minutes). 0.5N sodium hydroxide (4 mL) was added to the supernatant, and the absorbance was measured with an absorption spectrometer (560 nm). The gastric emptying rate was determined by the following calculation formula.

Gastric emptying rate(%)=(1−absorbance of test sample/absorbance of standard sample)×100

For absorbance of standard sample, the stomach isolated immediately after administration of 0.05% phenol red solution was used.

### Experiment Results.

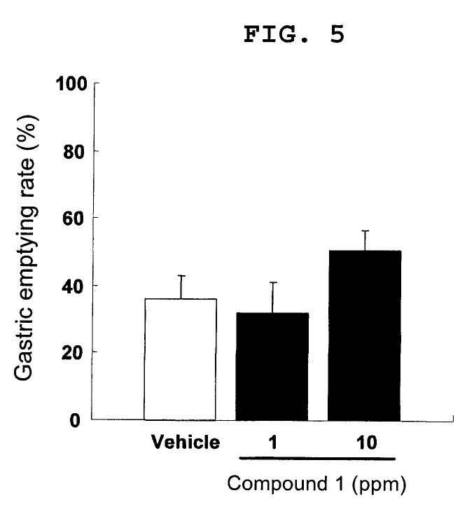

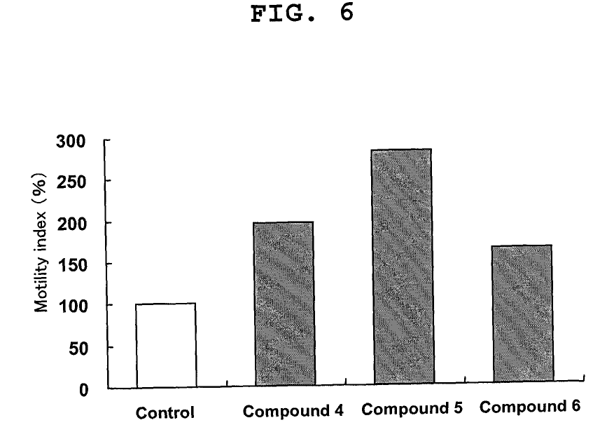

The results are shown in FIG. 4 and FIG. 5. As shown in the data, the tested group showed increase in the gastric emptying rate as compared to the control group.

### Example 5

### Gastrointestinal Motility Test Using T1R Agonist

### Experiment Method.

### Method for Measuring Gastrointestinal Movement Using Awaken Dog.

Female beagles, fasted for one night, were subjected to an abdominal operation under anesthesia with 1.0% isoflurane; a transducer for measuring gastrointestinal movement was sutured onto the stomach in the direction permitting measurement of annular muscle contraction; and the abdomen was closed. Not less than two weeks after the surgery, gastrointestinal movement was measured after one night-fasting; and 300 ml of condensed-fluid-diet (Ajinomoto Co. Inc., “MEDIEF BAG”) containing test compound (T1R agonist) 4, 5, or 6 described in the following Production Example (10 weight ppm, respectively) was orally given at 10 to 20 minutes after completion of the phase III of fasting-strong contraction movement (n=1 for each tested group). Motility index was calculated as the area surrounded by the baseline and a contraction wavy line from 60 to 90 minutes after the oral intake and represented as percentage against control group, to which only condensed-fluid-diet without the compound was orally given. Experiment Results.

The results are shown in FIG. 6. As shown in the data, the tested group showed increase in the gastrointestinal movement as compared to the control group.

### Example 6

### Stomach Content Emptying Test Using T1R Agonist

### Experiment Method.

### Mouse Gastric Emptying Method

Male ICR mice were used. A 5% casein fluid diet (0.5 mL) containing 0.05% phenol red and a test drug (3.5 mM Cyclamate, 3.7 mM MSG (monosodium glutamate), compound 2, 3, 4, or 5 (10 weight ppm, respectively) of the following Production Example) was orally administered, and 30 minutes later, the chest was opened and the stomach was isolated. The stomach was placed in 0.1N sodium hydroxide (14 mL), homogenized and left standing for 1 hour at room temperature. 20% Trichloroacetic acid (0.5 mL) was added to 5 mL of the supernatant, and the mixture was centrifuged (3000 rpm, 20 minutes). 0.5N sodium hydroxide (4 mL) was added to the supernatant, and the absorbance was measured with an absorption spectrometer (560 nm). The gastric emptying rate was determined by the following calculation formula.

Gastric emptying rate(%)=(1−absorbance of test sample/absorbance of standard sample)×100

For absorbance of standard sample, the stomach isolated immediately after administration of 0.05% phenol red solution was used.

### Experiment Results.

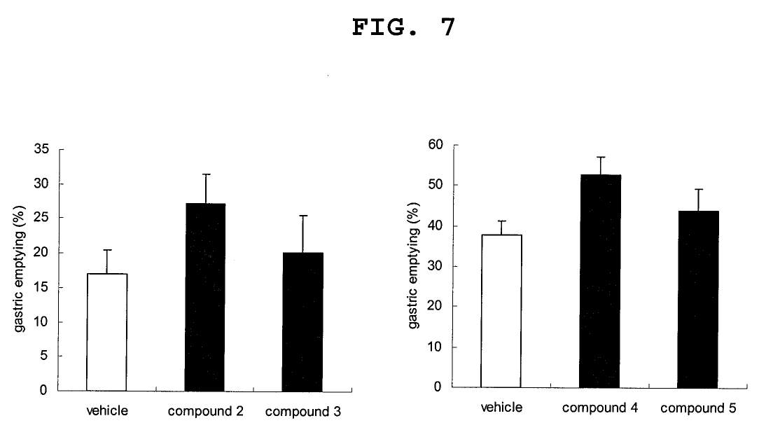

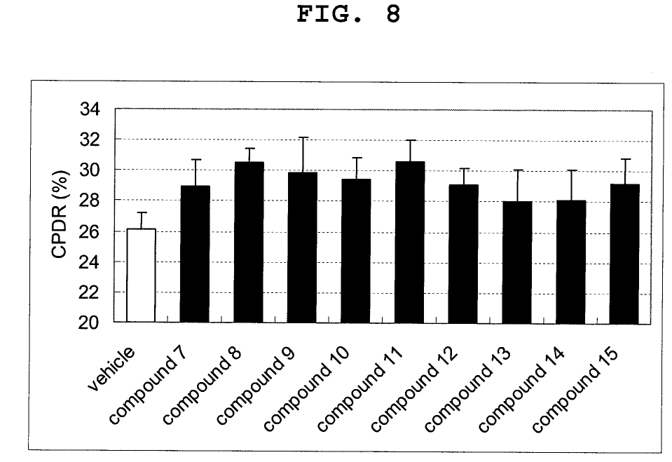

The results are shown in FIG. 7, where the vertical axis shows gastric emptying rate (%). As is clear from the Figure, compounds 2-5 promoted gastric emptying.

### Example 7

### ¹³C-Acetic Acid Breath Test

### Experiment Method.

Five healthy beagle dogs, weighting to 10 to 12 kg, were used in present study. The dogs were fed with 300 kcal/300 ml liquid meal (casein calcium 41.4 g, maltodextrin 37.5 g) containing 50 mg of ¹³C-acetic acid (Cambridge Laboratories, Boston, Mass., USA). Breath samples were continuously collected via a nasal tube using Breath ID system (Oridion Medical, Jerusalem, Israel) for 4 hours. The data were analyzed using Oridion Research Software (Oridion Medical) and calculated with Cumulative Percentage Dose Recovery (CPDR). The compounds 7 to 15 dissolved in the liquid meal, and were orally administered (3 mg/dog, in 300 ml liquid meal, respectively).

### Experiment Results.

CPDR (4 hours) represents amounts of ¹³CO₂ from breath samples for 4 hours, means gastric emptying volume for 4 hours. The results are shown in FIG. 8. As is clear from the figure, CPDR was increased by administrating compounds 7 to 15, suggested that compound 7 to 15 promoted the gastric emptying.

### Production Examples

Compounds 1-6 described in the following Table 3 and Compounds 7-15 described in the following table 4 were synthesized by the method described in the following Examples. However, the synthesis methods of these compounds are not limited to those in the following Examples. The structures of the compounds synthesized in the following Examples were identified by magnetic resonance spectrum (Bruker AVANCE400 (400 MHz)) and mass analysis (Thermo Quest TSQ700). Production Example 1. Synthesis of 3,6-dichloro-N-(4-ethoxyphenyl)-2-methoxybenzamide (compound 1).

To acetonitrile (30 mL) were added 3,6-dichloro-2-methoxybenzoic acid (1.68 g, 7.60 mmol) and p-phenetidine (1.04 g, 7.60 mmol). To this reaction mixture were added 1H-benzotriazol-1-yloxytris(dimethylamino)phosphonium hexafluorophosphate (BOP, 3.21 g, 7.60 mmol) and N,N-diisopropylethylamine (DIEA, 4.0 mL, 23.5 mmol), and the reaction mixture was stirred overnight at room temperature. The reaction mixture was concentrated under reduced pressure, ethyl acetate (100 mL) was added to the residue, and the mixture was stirred. The organic layer was washed with water (50 mL), aqueous 2N-hydrochloric acid solution (50 mL×2), saturated brine (50 mL), saturated aqueous sodium hydrogencarbonate solution (50 mL×2) and saturated brine (50 mL), and dried over anhydrous magnesium sulfate. Magnesium sulfate was removed by filtration, and the filtrate was concentrated under reduced pressure. The obtained residue was recrystallized twice from ethyl acetate-n-hexane to give the title compound (1.11 g, 3.26 mmol, 42.9%) as white crystals.

¹H-NMR (CDCl₃, δ): 1.44 (t, J=7.0 Hz, 3H), 3.99 (s, 3H), 4.03 (q, J=7.0 Hz, 2H), 6.90 (d, J=9.0 Hz, 2H), 7.15 (d, J=8.6 Hz, 1H), 7.40 (br.s, 1H), 7.36 (d, J=8.6 Hz, 1H), 7.50 (d, J=9.0 Hz, 2H).

ESI-MS: 340.2, 342.2 (M+H)⁺.

### Production Example 2

### Synthesis of 2,5-dichloro-N-(4-ethoxyphenyl)benzamide (compound 2)

In the same manner as in Production Example 1, except that 2,5-dichlorobenzoic acid was used instead of 3,6-dichloro-2-methoxybenzoic acid, the title compound was obtained as white crystals (yield 66.8%).

¹H-NMR (CDCl₃, δ): 1.42 (t, J=7.0 Hz, 3H), 4.04 (q, J=7.0 Hz, 2H), 6.90 (d, J=10.2 Hz, 2H), 7.38 (s, 2H), 7.51 (d, J=10.2 Hz, 2H), 7.74 (s, 1H), 7.78 (br.s, 1H).

ESI-MS: 310.2, 312.2 (M+H)⁺.

### Production Example 3

### Synthesis of N-(1-ethylpropyl)-benzofuran-2-carboxamide (compound 3)

In the same manner as in Production Example 1, except that benzofuran-2-carboxylic acid was used instead of 3,6-dichloro-2-methoxybenzoic acid and 3-aminopentane was used instead of p-phenetidine, the title compound was obtained as white crystals (yield 75.1%).

¹H-NMR (CDCl₃, δ): 1.03 (t, J=7.4 Hz, 6H), 1.48-1.59 (m, 2H), 1.64-1.75 (m, 2H), 3.98-4.07 (m, 1H), 6.35 (br.d, 1H), 7.26-7.31 (m, 1H), 7.38-7.43 (m, 1H), 7.46 (s, 1H), 7.50 (d, J=8.4 Hz, 1H), 7.68 (d, J=8.4 Hz, 1H).

ESI-MS: 232.0 (M+H)⁺.

### Production Example 4

### Synthesis of N-(1,2,3,4-tetrahydronaphthalen-1-yl)-benzo[1,3]dioxol-5-carboxamide (compound 4)

In the same manner as in Production Example 1, except that piperonylic acid was used instead of 3,6-dichloro-2-methoxybenzoic acid and 1,2,3,4-tetrahydro-1-naphthylamine was used instead of p-phenetidine, the title compound was obtained as white crystals (yield 74.7%).

¹H-NMR (CDCl₃, δ): 1.84-1.96 (m, 3H), 2.10-2.17 (m, 1H), 2.76-2.88 (m, 2H), 5.33-5.37 (m, 1H), 6.01 (s, 2H), 6.20 (br.d, 1H), 6.80 (d, J=8.5 Hz, 1H), 7.12-7.33 (m, 6H).

ESI-MS: 296.0 (M+H)⁺.

### Production Example 5

### Synthesis of 4-ethoxy-N-(1-propylbutyl)benzamide (compound 5)

To methylene chloride (30 mL) were added 4-ethoxybenzoic acid (1.66 g, 10.0 mmol) and 4-heptylamine (1.15 g, 10.0 mmol), and the solution was maintained at 0° C. in an ice bath. To the reaction mixture were added 1-hydroxybenzotriazole (HOBt, 1.68 g, 11.0 mmol) and 1-ethyl-3-(3-dimethylaminopropyl)carbodiimide hydrochloride (EDC, 2.11 g, 11.0 mmol), and the mixture was removed from the ice bath and stirred overnight at room temperature. The reaction mixture was concentrated under reduced pressure, ethyl acetate (100 mL) was added to the residue, and the mixture was stirred. The organic layer was washed with water (50 mL), 5% aqueous citric acid solution (50 mL×2), saturated brine (50 mL), 5% aqueous sodium hydrogencarbonate solution (50 mL×2), and saturated brine (50 mL), and dried over anhydrous magnesium sulfate. Magnesium sulfate was removed by filtration, and the filtrate was concentrated under reduced pressure. The obtained residue was recrystallized twice from ethyl acetate to give the title compound (663 mg, 2.52 mmol, 25.2%) as white crystals.

¹H-NMR (CDCl₃, δ): 0.90 (t, 7.1 Hz, 6H), 1.33-1.65 (m, 15H), 4.07 (q, J=7.0 Hz, 2H), 4.10-4.20 (m, 1H), 5.65-5.75 (m, 1H), 6.90 (d, J=8.8 Hz, 2H), 7.71 (d, J=8.8 Hz, 2H).

ESI-MS: 264.0 (M+H)⁺.

### Production Example 6

### Synthesis of 3-(4-methoxyphenyl)-N-(1-propylbutyl)acrylamide (compound 6)

In the same manner as in Production Example 5, except that 4-methoxy cinnamic acid was used instead of 4-ethoxybenzoic acid, the title compound was obtained as white crystals (yield 41.4%).

¹H-NMR (CDCl₃, δ): 0.90 (t, J=7.0 Hz, 6H), 1.30-1.55 (m, 12H), 3.82 (s, 3H), 4.05-4.15 (m, 1H), 5.35-5.55 (m, 1H), 6.27 (d, J=15.5 Hz, 1H), 6.87 (d, J=8.8 Hz, 2H), 7.43 (d, J=8.8 Hz, 2H), 7.58 (d, J=15.5 Hz, 1H).

ESI-MS: 276.1 (M+H)⁺.

TABLE 3

| | Structure | Name |
|---|---|---|
| compound 1 | 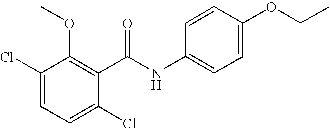 | 3,6-dichloro-N-(4-ethoxyphenyl)-2-methoxybenzamide |
| compound 2 | 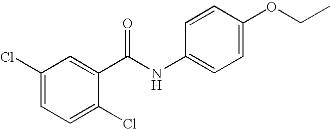 | 2,5-dichloro-N-(4-ethoxyphenyl)-benzamide |
| compound 3 | 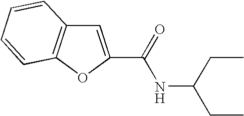 | N-(1-ethylpropyl)-benzofuran-2-carboxamide |
| compound 4 | 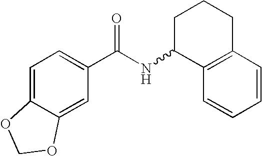 | N-(1,2,3,4-tetrahydronaphthalen-1-yl)-benzo[1,3]dioxol-5-carboxamide |
| compound 5 | 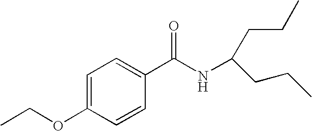 | 4-ethoxy-N-(1-propylbutyl)-benzamide |
| compound 6 | 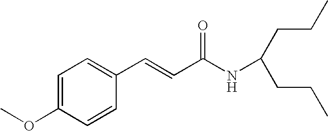 | 3-(4-methoxyphenyl)-N-(1-propylbutyl)acrylamide |

### Production Example 7

### Synthesis of N-(2,4-dimethoxybenzyl)-N′-(2-(5-methylpyridin-2-yl)ethyl)oxalamide Hydrochloride (compound 7)

To a solution of 2,4-dimethoxybenzylamine (5.50 g) in 1,4-dioxane (120 mL) were added triethylamine (5.50 mL) and ethyl oxalyl chloride (4.04 mL) and stirred for 1 hour at room temperature. The mixture was evaporated under reduced pressure. To the residue was added water, and extraction with AcOEt was conducted. The organic phase was washed successively with aqeuous 1M HCl, saturated aqueous NaCl, dried over MgSO₄, and evaporated under reduced pressure to give ethyl 2-(2,4-dimethoxybenzylamino)-2-oxoacetate (9.37 g).

A mixture of ethyl 2-(2,4-dimethoxybenzylamino)-2-oxoacetate (1.0 g), 2-(5-methylpyridin-2-yl)ethylamine (1.0 g), triethylamine (1.04 mL) and 1,4-dioxane (20 mL) was stirred for 15 hours at 80° C. and was evaporated under reduced pressure. This reaction was conducted again in a similar manner using 2-(2,4-dimethoxybenzylamino)-2-oxoacetate (2.22 g), 2-(5-methylpyridin-2-yl)ethylamine (1.13 g), triethylamine (2.31 mL) and 1,4-dioxane (20 mL). These crude products were combined and purified by silica gel chromatography to afford a free form of N-(2,4-dimethoxybenzyl)-N′-(2-(5-methylpyridin-2-yl)ethyl)oxalamide as a solid (0.89 g). To a solution of the free form (0.78 g) in CHCl₃ (25 mL) was added 4N HCl in 1,4-dioxane (2.5 mL) and the mixture was evaporated under reduced pressure and dried in vacuo to give the HCl salt as a solid (0.83 g).

¹H-NMR (CDCl₃, δ): 2.52 (s, 3H), 3.50 (t, J=6.6 Hz, 2H), 3.80 (s, 3H), 3.83 (s, 3H), 3.89 (q, J=6.6 Hz, 2H), 4.38 (d, J=6.3 Hz, 2H), 6.40-6.47 (m, 2H), 7.14 (d, J=7.8 Hz, 1H), 7.58 (d, J=8.4 Hz, 1H), 7.67-7.73 (m, 1H), 7.96-8.01 (m, 1H), 8.04 (dd, J=7.8 Hz, 1.5 Hz, 1H), 8.51 (s, 1H).

ESI-MS: 358 (M+H)⁺.

### Production Example 8

### Synthesis of N-(2-chlorobenzyl)-N′-(2-(pyridin-2-yl)ethyl)oxalamide Hydrochloride (compound 8)

To a solution of 2-chlorobenzylamine (1.21 mL) in 1,4-dioxane (50 mL) were added triethylamine (2.79 mL) and ethyl oxalyl chloride (1.12 mL) and stirred for 1.5 hours at room temperature. To the reaction mixture was added 2-(2-aminoethyl)pyridine (1.20 mL) and stirred at 80° C. overnight. The mixture was evaporated under reduced pressure. To the residue was added water and extraction with AcOEt was conducted. The organic phase was washed with saturated aqueous NaCl, dried over MgSO₄, and filtered. During an evaporation of the filtrate under reduced pressure, a precipitate produced. It was filtered and washed with Et₂O to afford a free form of N-(2,4-dimethoxybenzyl)-N′-(2-(5-methylpyridin-2-yl)ethyl)oxalamide as a solid (1.3 g). The free form was dissolved with AcOEt-MeOH (20 mL, 4 ml) and to the solution was added 4N HCl in AcOEt (5 mL). The produced precipitate was filtered, washed with AcOEt, and dried at 60° C. in vacuum to the HCl salt as a solid (1.26 g).

¹H-NMR (D₂O, δ): 3.19 (t, 2H, J=6.3 Hz), 3.63 (t, 2H, J=6.3 Hz), 4.40 (s, 2H), 7.18-7.24 (m, 3H), 7.32-7.38 (m, 1H), 7.75-7.82 (m, 2H), 8.35 (dt, J=8.4 Hz, 1.8 Hz, 1H), 8.49-8.52 (m, 1H).

ESI-MS: 318 (M+H)⁺.

### Production Example 9

### Synthesis of 2-((5-(2-methoxy-4-methylphenyl)-1H-1,2,4-triazol-3-ylthio)methyl)pyridine Hydrochloride (compound 9)

To a solution of 2-methoxy-4-methylbenzoic acid (3.62 g) in pyridine (18 mL) was added 1-ethyl-3-(3-dimethylaminopropyl)carbodiimide hydrochloride (EDC, 4.38 g) in pyridine (18 mL) was added thiosemicarbazide (1.89 g) and was stirred for 19 hours at room temperature. The mixture was evaporated under reduced pressure and eluted with water (20 mL). The solid was filtered, washed with water, and suspended in 1M Na₂CO₃ aqueous solution (30 mL). The mixture was stirred at 100° C. in a sealed tube for 23 hours. To the mixture was added AcOEt (200 mL) and water (100 ml). The organic phase was separated, dried over Na₂SO₄ and concentrated under reduced pressure to give 5-(2-methoxy-4-methyl)-2H-1,2,4-triazole-3(4H)-thione as a solid (0.39 g). A mixture of 5-(2-methoxy-4-methyl)-2H-1,2,4-triazole-3(4H)-thione (0.29 g), 2-(bromomethyl)pyridine hydrobromide (0.40 g) and ethanol (5.2 mL) was stirred for 23 hours at 60° C. To the mixture was added AcOEt (200 mL) and water (100 ml). The organic phase was separated, washed with NaHCO₃ aqueous solution, dried over Na₂SO₄ and concentrated under reduced pressure. The residue was purified with silica gel chromatography to give a free form of 2-((5-(2-methoxy-4-methylphenyl)-1H-1,2,4-triazol-3-ylthio)methyl)pyridine as a solid (0.26 g). It was dissolved in CH₂Cl₂ and to the solution was added 4M HCl 1,4-dioxane solution (1 mL) and stirred at room temperature. The mixture was concentrated under reduced pressure to give the HCl salt as a solid (0.30 g).

¹H-NMR (DMSO-d₆, δ): 2.37 (s, 3H), 3.91 (s, 3H), 4.77 (s, 2H), 6.88-6.93 (m, 1H), 7.02 (s, 1H), 7.84-7.91 (m, 2H), 8.06 (d, J=8.1 Hz, 1H), 8.44 (dt, J=7.8 Hz, 1.8 Hz, 1H), 8.82-8.86 (m, 1H)

ESI-MS: 313 (M+H)⁺.

### Production Example 10

### Synthesis of 2-((5-(2,4-dimethylphenyl)-1H-1,2,4-triazol-3-ylthio)methyl)pyridine Hydrochloride (compound 10)

In the same manner as in Production Example 9, except that 2,4-dimethylbenzoic acid was used instead of 2-methoxy-4-methylbenzoic acid, the title compound was obtained as a solid (2.30 g).

¹H-NMR (DMSO-d₆, δ): 2.31 (s, 3H), 2.49 (s, 3H), 4.77 (s, 2H), 7.09-7.18 (m, 2H), 7.54 (d, J=8.1 Hz, 1H), 7.84-7.91 (m, 1H), 8.04 (d, J=7.8 Hz, 1H), 8.45 (dt, J=8.1 Hz, 1.8 Hz, 1H), 8.81-8.85 (m, 1H).

ESI-MS: 297 (M+H)⁺.

### Production Example 11

### Synthesis of 2-amino-3-methoxy-N-(5-methoxy-2,3-dihydro-1H-inden-1-yl)benzamide Hydrochloride (compound 11)

To a mixture of 2,3-dihydro-5-methoxy-1H-inden-1-amine (0.76 g), 2-amino-3-methoxy benzoic acid (0.93 g), 1-hydroxybenzotriazole (HOBt, 0.86 g) and DMF (15 mL) cooled with ice were added triethylamine (1.5 mL) and 1-ethyl-3-(3-dimethylaminopropyl)carbodiimide hydrochloride (EDC, 1.79 g) and stirred overnight at 40° C. The mixture was poured into water and extracted with AcOEt. The organic phase was washed successively with water, saturated NaCl aq., dried over Na₂SO₄, and evaporated under reduced pressure. The residue was purified with silica gel chromatography to give a free form of 2-amino-3-methoxy-N-(5-methoxy-2,3-dihydro-1H-inden-1-yl)benzamide (0.80 g). To a solution of the free form in 1,4-dioxane (10 mL) was added 4N HCl in 1,4-dioxane (5 mL) and stirred overnight at room temperature. The precipitate was filtered, washed with Et₂O, and dried in vacuo to give the HCl salt as a solid (0.75 g).

¹H-NMR (DMSO-d₆, δ): 1.90-1.96 (m, 2H), 2.78 (t, J=6.6 Hz, 2H), 3.68 (s, 3H), 3.71 (t, J=6.3 Hz, 1H), 3.85 (s, 3H), 6.50-6.55 (m, 2H), 6.75-6.82 (m, 2H), 6.87 (d, J=9.0 Hz, 1H), 7.00 (dd, J=8.4 Hz, 1.2 Hz, 1H).

ESI-MS: 313 (M+H)⁺.

### Production Example 12

### Synthesis of 2-amino-3-methoxy-N-(6-methoxy-1,2,3,4-tetrahydronaphthalen-1-yl)benzamide Hydrochloride (compound 12)

In the same manner as in Production Example 11, except that 1,2,3,4-tetrahydro-6-methoxy-1-naphthalenamine was used instead of 2,3-dihydro-5-methoxy-1H-inden-1-amine, the title compound was obtained as a solid (0.44 g).

¹H-NMR (DMSO-d₆, δ): 1.31-1.50 (m, 1H), 1.70-2.08 (m, 3H), 2.55-2.72 (m, 1H), 2.75-3.00 (m, 2H), 3.67 (s, 3H), 3.80 (s, 3H), 4.80 (br.d, 1H), 6.23 (d, J=7.8 Hz, 1H), 6.49 (d, J=8.1 Hz, 1H), 6.63 (t, J=7.5 Hz, 1H), 6.77 (d, J=8.1 Hz, 1H), 6.80-6.92 (m, 2H).

ESI-MS: 327 (M+H)⁺.

### Production Example 13

### Synthesis of N-(heptan-4-yl)-1,2,3,4-tetrahydroquinoline-7-carboxamide Hydrochloride (compound 13)

A mixture of 1,2,3,4-tetrahydroquinoline-7-carboxylic acid (1.04 g), di-tert-butyl dicarbonate (1.92 g), 1N NaOH aqueous solution (20 mL) and 1,4-dioxane (20 mL) was stirred at room temperature overnight. To the mixture cooled with ice was added 2N HCl aqueous solution (13.5 mL) and extraction was conducted with AcOEt. The organic phase was dried over MgSO₄ and evaporated under reduced pressure. The residue was dissolved in CH₂Cl₂-DMF (20 mL-15 mL) and then to the solution was added 2-(1H-7-azabenzotriazol-1-yl)-1,1,3,3-tetramethyluronium hexafluorophosphate methanaminium (HATU, 2.69 g), 1-hydroxy-7-azabenzotriazole (HOAt, 0.96 g), di-isopropyl-ethylamine (1.75 g) and 4-heptylamine (0.81 g) and stirred for 1.5 hours at room temperature. It was poured into saturated NaHCO₃ aqueous solution and extracted with AcOEt. The organic phase was dried over MgSO₄ and evaporated under reduced pressure. The crude product was purified by silica gel chromatography to afford a free form of N-(heptan-4-yl)-1,2,3,4-tetrahydroquinoline-7-carboxamide. It was dissolved in CH₂Cl₂ (10 mL) and to the solution was added 4N HCl in 1,4-dioxane (4 mL) followed by evaporation under reduced pressure. The residue was purified by preparative HPLC to remove a small amount of impurities. It was again dissolved in CH₂Cl₂ (10 mL) and to the solution was added 4N HCl in 1,4-dioxane (4 mL) followed by evaporation under reduced pressure. The residue was suspended with Et₂O, evaporated and dried in vacuo to give the HCl salt as a solid (0.22 g).

¹H-NMR (DMSO-d₆, δ): 0.84 (t, J=7.2 Hz, 6H), 1.18-1.43 (m, 4H), 1.45-1.50 (m, 4H), 1.90-2.02 (m, 2H), 2.81 (t, J=6.3 Hz, 1H), 3.28-3.33 (m, 2H), 3.90-4.00 (m, 1H), 7.25 (d, J=7.8 Hz, 1H), 7.55 (s, 1H), 7.62 (d, J=8.1 Hz, 1H), 8.06 (d, J=8.7 Hz, 1H).

ESI-MS: 275 (M+H)⁺.

### Production Example 14

### Synthesis of (R)-methyl 2-(3-chloro-4-methoxybenzamido)-4-methylpentanoate (compound 14)

To a mixture of 3-chloro-4-methoxybenzoic acid (1.69 g), D-leucine methyl ester hydrochloride salt (1.50 g), 1-hydroxybenzotriazole (HOBt, 1.39 g) and DMF (15 mL) cooled with ice were added triethylamine (3.45 mL) and 1-ethyl-3-(3-dimethylaminopropyl)carbodiimide hydrochloride (EDC, 3.17 g) and stirred for 22 hours at room temperature. The mixture was poured into water (90 mL) and extracted with AcOEt. The organic phase was washed successively with 1N KOH aq., 1N HCl aq., saturated NaCl aq., dried over Na₂SO₄, and evaporated under reduced pressure. The residue was purified with silica gel chromatography to give the titled compound (2.66 g).

¹H-NMR (CDCl₃, δ): 0.96-1.01 (m, 6H), 1.62-1.80 (m, 3H), 3.78 (s, 3H), 3.95 (s, 3H), 4.80-4.88 (m, 1H), 6.45 (br.d, 1H), 6.95 (d, J=8.4 Hz, 1H), 7.71 (dd, J=8.4 Hz, 2.4 Hz, 1H), 7.82 (d, J=2.1 Hz, 1H).

ESI-MS: 314 (M+H)⁺.

### Production Example 15

### Synthesis of (R)-methyl 4-methyl-2-(4-methylthio)-benzamido)pentanoate (compound 15)

To a solution of D-leucine methyl ester hydrochloride salt (1.0 g) in DMF (10 ml) and triethylamine (1.87 mL) cooled with ice was added 4-methylthiobenzoyl chloride (1.00 g) and stirred for 15 hours at room temperature. To the mixture was added AcOEt (200 mL) and water (100 mL). The organic phase was separated, washed successively with 1N KOH aq., 1N HCl aq., saturated NaCl aq., dried over Na₂SO₄ and evaporated under reduced pressure. The residue was purified with silica gel chromatography to give the titled compound (1.34 g).

¹H-NMR (CDCl₃, δ): 0.96-1.01 (m, 6H), 1.62-1.80 (m, 3H), 2.51 (s, 3H), 3.76 (s, 3H), 4.82-4.90 (m, 1H), 6.51 (br.d, 1H), 7.23-7.28 (m, 2H), 7.69-7.74 (m, 2H).

ESI-MS: 296 (M+H)⁺.

TABLE 4

| | Structure | Name |
|---|---|---|
| compound 7 | 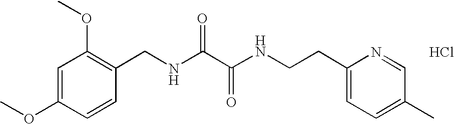 | N-(2,4-dimethoxybenzyl)-N′-(2-(5-methylpyridin-2-yl)ethyl)oxalamide HCl |
| compound 8 | 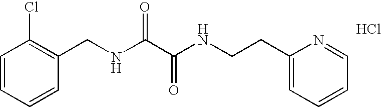 | N-(2-chlorobenzyl)-N′-(2-(pyridin-2-yl)ethyl)oxalamideHCl |
| compound 9 | 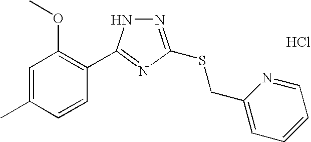 | 2-((5-(2-methoxy-4-methylphenyl)-1H-1,2,4-triazol-3-ylthio) methyl)pyridine HCl |
| compound 10 | 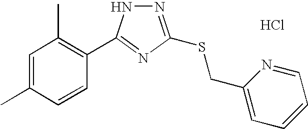 | 2-((5-2,4-dimethylphenyl)-1H-1,2,4-triazol-3-ylthio)methyl)pyridine HCl |
| compound 11 | 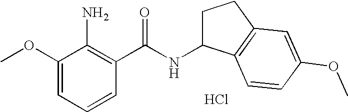 | 2-amino-3-methoxy-N-(5-methoxy-2,3-dihydro-1H-inden-1-yl)benzamide HCl |
| compound 12 | 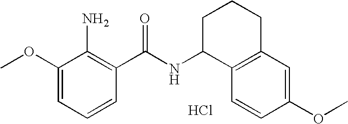 | 2-amino-3-methoxy-N-(6-methoxy-1,2,3,4-tetrahydronaphthalen-1-yl)benzamide HCl |
| compound 13 | 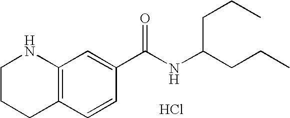 | N-(heptan-4-yl)-1,2,3,4-tetrahydroquinoline-7-carboxamide HCl |
| compound 14 | 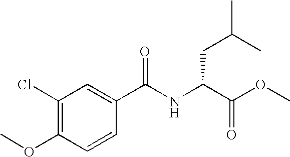 | (R)-methyl 2-(3-chloro-4-methoxybenzamido)-4-methylpentanoate |
| compound 15 | 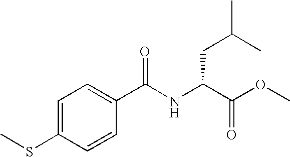 | (R)-methyl 4-methyl-2-(4-(methylthio)benzamido)-pentanoate |

### INDUSTRIAL APPLICABILITY

According to the present invention, a pharmaceutical agent, food, drink, or method for the promotion of gastrointestinal function, which is useful for the prophylaxis or improvement of, for example, functional gastrointestinal disorders, particularly upper gastrointestinal dysfunctions such as functional dyspepsia, gastroesophageal reflux disease and the like can be provided. Using the composition of the present invention, prophylaxis or improvement of dyspepsia associated with gastrointestinal dysfunction such as FD and the like can be achieved safely and effectively, without inducing side effects. Moreover, the screening method of the present invention is used for the detection of an active ingredient useful for the above-mentioned pharmaceutical agent, food, drink, or method of the present invention, as well as usable for experiments in the fields of physiology.biochemistry.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method of promoting a gastrointestinal function, which comprises administering an effective amount of at least one T1R agonist to a mammal in need thereof.

2. The method of claim 1, wherein said at least one T1R agonist is Cyclamate.

3. The method of claim 1, wherein said at least one T1R agonist is a compound represented by formula (I): 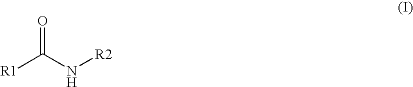
wherein:
R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,
R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and
R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),
or a pharmacologically acceptable salt thereof.

4. The method of claim 3, wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

5. The method of claim 3, wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

6. The method of claim 1, wherein said at least one T1R agonist is a compound represented by formula (II):
Ar1-Y-hAr1-X—(CR10R11)ₙ-hAr2 (II)
wherein Ar1 is an aryl group optionally having substituent(s) or a heteroaryl group optionally having substituent(s),
Y is single bond, O, S, S(O), SO₂, CR4R5 or NR6,
hAr1 is a heteroaryl group optionally having substituent(s),
X is O, S, SO, SO₂, CR7R8 or NR9,
n is an integer of 0 to 3,
hAr2 is an heteroaryl group optionally having substituent(s),
each of R4, R5, R7, R8, R10, and R11 is independently selected from hydrogen, oxygen, hydroxyl, NH₂, SH, halogen and C₁-C₄ organic group, and
each of R6 and R9 is independently selected from hydrogen, hydroxyl and C₁-C₄ organic group.

7. The method of claim 6, wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

8. The method of claim 1, which comprises administering a pharmaceutical composition comprising said at least one T1R agonist and a carrier to a mammal.

9. The method of claim 1, which comprises administering a food or drink comprising said at least one T1R agonist in an amount of 0.01 to 100,000 weight ppm to a mammal.

10. A method of promoting a gastrointestinal function, which comprises administering an effective amount of at least one T1R agonist to a mammal in need thereof, wherein the promotion of gastrointestinal function is improvement of a functional gastrointestinal disorder.

11. The method of claim 10, wherein said at least one T1R agonist is Cyclamate.

12. The method of claim 10, wherein said at least one T1R agonist is a compound represented by formula (I): 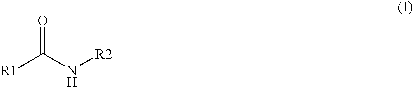
wherein:
R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,
R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and
R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),
or a pharmacologically acceptable salt thereof.

13. The method of claim 10, wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

14. The method of claim 10, wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

15. The method of claim 10, wherein said at least one T1R agonist is a compound represented by formula (II):
Ar1-Y-hAr1-X—(CR10R11)ₙ-hAr2 (II)
wherein Ar1 is an aryl group optionally having substituent(s) or a heteroaryl group optionally having substituent(s),
Y is single bond, O, S, S(O), SO₂, CR4R5 or NR6,
hAr1 is a heteroaryl group optionally having substituent(s),
X is O, S, SO, SO₂, CR7R8 or NR9,
n is an integer of 0 to 3,
hAr2 is an heteroaryl group optionally having substituent(s),
each of R4, R5, R7, R8, R10, and R11 is independently selected from hydrogen, oxygen, hydroxyl, NH₂, SH, halogen and C₁-C₄ organic group, and
each of R6 and R9 is independently selected from hydrogen, hydroxyl and C₁-C₄ organic group.

16. The method of claim 10, wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

17. The method of claim 10, which comprises administering a pharmaceutical composition comprising said at least one T1R agonist and a carrier to a mammal.

18. The method of claim 10, which comprises administering a food or drink comprising said at least one T1R agonist in an amount of 0.01 to 100,000 weight ppm to a mammal.

19. A method of promoting a gastrointestinal function, which comprises administering an effective amount of at least one T1R agonist to a mammal in need thereof, wherein the promotion of gastrointestinal function is improvement of an upper gastrointestinal dysfunction.

20. The method of claim 19, wherein said at least one T1R agonist is Cyclamate.

21. The method of claim 19, wherein said at least one T1R agonist is a compound represented by formula (I): 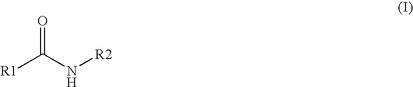
wherein:
R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,
R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and
R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),
or a pharmacologically acceptable salt thereof.

22. The method of claim 19, wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

23. The method of claim 19, wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

24. The method of claim 19, wherein said at least one T1R agonist is a compound represented by formula (II):
Ar1-Y-hAr1-X—(CR10R11)ₙ-hAr2 (II)
wherein Ar1 is an aryl group optionally having substituent(s) or a heteroaryl group optionally having substituent(s),
Y is single bond, O, S, S(O), SO₂, CR4R5 or NR6,
hAr1 is a heteroaryl group optionally having substituent(s),
X is O, S, SO, SO₂, CR7R8 or NR9,
n is an integer of 0 to 3,
hAr2 is an heteroaryl group optionally having substituent(s),
each of R4, R5, R7, R8, R10, and R11 is independently selected from hydrogen, oxygen, hydroxyl, NH₂, SH, halogen and C₁-C₄ organic group, and
each of R6 and R9 is independently selected from hydrogen, hydroxyl and C₁-C₄ organic group.

25. The method of claim 19, wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

26. The method of claim 19, which comprises administering a pharmaceutical composition comprising said at least one T1R agonist and a carrier to a mammal.

27. The method of claim 19, which comprises administering a food or drink comprising said at least one T1R agonist in an amount of 0.01 to 100,000 weight ppm to a mammal.

28. A method of promoting a gastrointestinal function, which comprises administering an effective amount of at least one T1R agonist to a mammal in need thereof, wherein the promotion of gastrointestinal function is improvement of functional dyspepsia or a gastroesophageal reflux disease.

29. The method of claim 28, wherein said at least one T1R agonist is Cyclamate.

30. The method of claim 28, wherein said at least one T1R agonist is a compound represented by formula (I): 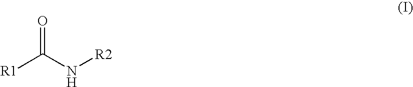
wherein:
R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,
R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and
R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),
or a pharmacologically acceptable salt thereof.

31. The method of claim 28, wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

32. The method of claim 28, wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

33. The method of claim 28, wherein said at least one T1R agonist is a compound represented by formula (II):
Ar1-Y-hAr1-X—(CR10R11)-hAr2 (II)
wherein Ar1 is an aryl group optionally having substituent(s) or a heteroaryl group optionally having substituent(s),
Y is single bond, O, S, S(O), SO₂, CR4R5 or NR6,
hAr1 is a heteroaryl group optionally having substituent(s),
X is O, S, SO, SO₂, CR7R8 or NR9,
n is an integer of 0 to 3,
hAr2 is an heteroaryl group optionally having substituent(s),
each of R4, R5, R7, R8, R10, and R11 is independently selected from hydrogen, oxygen, hydroxyl, NH₂, SH, halogen and C₁-C₄ organic group, and
each of R6 and R9 is independently selected from hydrogen, hydroxyl and C₁-C₄ organic group.

34. The method of claim 28, wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

35. The method of claim 28, which comprises administering a pharmaceutical composition comprising said at least one T1R agonist and a carrier to a mammal.

36. The method of claim 28, which comprises administering a food or drink comprising said at least one T1R agonist in an amount of 0.01 to 100,000 weight ppm to a mammal.

37. A method of regulating an appetite, which comprises administering an effective amount of at least one T1R agonist to a mammal in need thereof.

38. The method of claim 37, wherein said at least one T1R agonist is Cyclamate.

39. The method of claim 37, wherein said at least one T1R agonist is a compound represented by formula (I): 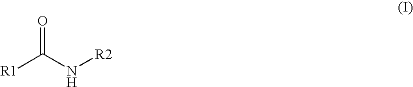
wherein:
R1 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s), a heteroarylalkenyl group optionally having substituent(s), R3-NH—CO— or R3-NH—,
R3 is an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s), and
R2 is a C₂₋₂₅ alkyl group optionally having substituent(s), a C₃₋₂₅ cycloalkyl group optionally having substituent(s) (said cycloalkyl group is optionally condensed with benzene), an aryl group optionally having substituent(s), an aralkyl group optionally having substituent(s), an arylalkenyl group optionally having substituent(s), a heteroaryl group optionally having substituent(s), a heteroaralkyl group optionally having substituent(s) or a heteroarylalkenyl group optionally having substituent(s),
or a pharmacologically acceptable salt thereof.

40. The method of claim 37, wherein said at least one T1R agonist is a compound selected from the group consisting of:
3,6-dichloro-N-(4-ethoxyphenyl)-2-methoxybenzamide,
2,5-dichloro-N-(4-ethoxyphenyl)benzamide,
N-(1-ethylpropyl)-benzofuran-2-carboxamide,
N-(1,2,3,4-tetrahydronaphthalen-1-yl)-benzo[1,3]dioxol-5-carboxamide,
4-ethoxy-N-(1-propylbutyl)benzamide, and
3-(4-methoxyphenyl)-N-(1-propylbutyl)acrylamide.

41. The method of claim 37, wherein said at least one T1R agonist is a compound selected from the group consisting of:
N-(2,4-dimethoxybenzyl)-N′-(2-(5-methylpyridin-2-yl)ethyl)oxalamide,
N-(2-chlorobenzyl)-N′-(2-(pyridin-2-yl)ethyl)oxalamide,
2-amino-3-methoxy-N-(5-methoxy-2,3-dihydro-1H-inden-1-yl)benzamide,
2-amino-3-methoxy-N-(6-methoxy-1,2,3,4-tetrahydronaphthalen-1-yl)benzamide,
N-(heptan-4-yl)-1,2,3,4-tetrahydroquinoline-7-carboxamide,
(R)-methyl 2-(3-chloro-4-methoxybenzamido)-4-methylpentanoate, and
(R)-methyl 4-methyl-2-(4-(methylthio)benzamido)pentanoate.

42. The method of claim 37, wherein said at least one T1R agonist is a compound represented by formula (II):
Ar1-Y-hAr1-X—(CR10R11)ₙ-hAr2 (II)
wherein Ar1 is an aryl group optionally having substituent(s) or a heteroaryl group optionally having substituent(s),
Y is single bond, O, S, S(O), SO₂, CR4R5 or NR6,
hAr1 is a heteroaryl group optionally having substituent(s),
X is O, S, SO, SO₂, CR7R8 or NR9,
n is an integer of 0 to 3,
hAr2 is an heteroaryl group optionally having substituent(s),
each of R4, R5, R7, R8, R10, and R11 is independently selected from hydrogen, oxygen, hydroxyl, NH₂, SH, halogen and C₁-C₄ organic group, and
each of R6 and R9 is independently selected from hydrogen, hydroxyl and C₁-C₄ organic group.

43. The method of claim 37, wherein said at least one T1R agonist is a compound selected from the group consisting of:
2-((5-(2-methoxy-4-methylphenyl)-1H-1,2,4-triazol-3-ylthio)methyl)pyridine, and
2-((5-(2,4-dimethylphenyl)-1H-1,2,4-triazol-3-ylthio)methyl)pyridine.

44. The method of claim 37, which comprises administering a pharmaceutical composition comprising said at least one T1R agonist and a carrier to a mammal.

45. The method of claim 37, which comprises administering a food or drink comprising said at least one T1R agonist in an amount of 0.01 to 100,000 weight ppm to a mammal.

46. A method of screening for a substance capable of promoting a gastrointestinal function, which uses a cell expressing a T1R receptor.

47. The method of claim 46, wherein the substance capable of promoting a gastrointestinal function is a T1R agonist or T1R modulator.

48. A method of screening for a substance capable of promoting a gastrointestinal function, which comprises:
(a) contacting a test substance with a cell expressing a T1R receptor,
(b) determining the activation of G protein in the cell contacted with said test substance, and comparing the activation with that of a control cell free of a contact with said test substance, and
(c) selecting a substance capable of promoting a gastrointestinal function, based on the comparison results of the step (b).

49. The method of claim 48, wherein an index for determining the activation of G protein is selected from an intracellular calcium concentration, an intracellular cAMP amount, an extracellular proton amount, and an intracellular gastrointestinal hormone secretory amount.

50. A method of screening for a substance capable of promoting a gastrointestinal function, which comprises:
(a) contacting a test substance and a ligand acting on T1R receptor with a cell expressing a T1R receptor,
(b) measuring the amount of said ligand bound with a cell membrane of the cell, and comparing the amount with that of a control cell free of a contact with said test substance, and
(c) selecting a substance capable of promoting a gastrointestinal function, based on the comparison results of the step (b).


SEQUENCE LISTING (2 sequences)

SEQ ID NO: 1 - DNA, 23 bp, Artificial Sequence
  Synthetic Construct
aggaccaccgtggtcgtggtctt

SEQ ID NO: 2 - DNA, 23 bp, Artificial Sequence
  Synthetic Construct
gcactcaagaatcaccagatggg